(19) United States
(12) Patent Application Publication
(10) Pub. No.: US 2026/0047886 A1
(43) Pub. Date: Feb. 19, 2026

(54) SYSTEMS AND METHODS FOR SELECTING DEVICE FOR PLACEMENT WITHIN AN ANATOMICAL STRUCTURE ON AN ULTRASOUND IMAGE FEED

(71) Applicant: Clarius Mobile Health Corp., Vancouver (CA)

(72) Inventors: Kris Dickie, Vancouver (CA); Laurent Pelissier, North Vancouver (CA); Sarah Leverett, Scottsdale, AZ (US)

(21) Appl. No.: 18/807,311

(22) Filed: Aug. 16, 2024

(51) Int. Cl. A61B 34/10; G06T 7/12; G06T 7/62; G06V 10/70; G06V 20/50; G16H 40/67

(52) U.S. Cl. CPC A61B 34/10; G06T 7/12; G06T 7/62; G06V 10/70; G06V 20/50; G16H 40/67; A61B 2034/105; A61B 2034/108; G06T 2207/10132; G06T 2207/20081; G06T 2207/20084; G06V 2201/03

(57) ABSTRACT

A method and system of selecting from a plurality of devices for placement within an anatomical structure on an ultrasound image feed that is acquired from an ultrasound scanner, the method comprising: displaying, on a screen communicatively connected to the ultrasound scanner, the ultrasound image feed comprising the anatomical structure; deploying an AI model to execute on a computing device communicatively connected to the ultrasound scanner, wherein the AI model is trained so that when the AI model is deployed, the computing device identifies and predicts one or more dimensions of the anatomical structure; acquiring, at the computing device, a new ultrasound image during ultrasound scanning; processing, using the AI model, the new ultrasound image to identify and predict the one or more dimensions of the anatomical structure; and automatically selecting a device from the plurality of devices for placement therein based on the one or more dimensions.

### FIELD OF THE INVENTION

The present disclosure relates generally to ultrasound imaging, and in particular, systems and methods for selecting a device for placement within an anatomical structure on an ultrasound image feed.

### BACKGROUND OF THE INVENTION

Often, a device is required to be inserted or placed within an anatomical structure of a patient. Procedures such as endotracheal intubation or catheter insertion can lead to complications when the size of the device being inserted is not correctly fitted to the patient.

The device being placed within the anatomical structure is usually to fulfill various functions. Tracheal tubes, for example, are used for maintaining an airway and sealing the trachea to facilitate positive pressure ventilation and/or to protect the lungs from aspiration. When the device is improperly sized, those functions may not be accomplished properly and can lead to severe complications. Similarly, catheters that are too large or small can result in urethral trauma or leakage, or possibly improper drainage which can result in lengthier recovery time and/or infections.

Typically, the size of the devices is selected according to age and height-based formulas. Unfortunately, between different individuals, there is often a significant variation in size and shape of the anatomical structure such that the correlation between age, height, weight, body surface area and anatomical structure shape or size is poor.

There is, thus, a need for improved ultrasound systems and methods for selecting a device for placement within an anatomical structure. The embodiments discussed herein may address and/or ameliorate at least some of the aforementioned drawbacks identified above. The foregoing examples of the related art and limitations related thereto are intended to be illustrative and not exclusive. Other limitations of the related art will become apparent to those of skill in the art upon a reading of the specification and a study of the drawings herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting examples of various embodiments of the present disclosure will next be described in relation to the drawings, in which:

FIG. 1 is a schematic diagram of an ultrasound imaging system, according to an embodiment of the present invention;

FIG. 2 is a schematic diagram of a system with multiple ultrasound scanners, according to an embodiment of the present invention;

FIG. 3 is a flowchart diagram showing steps of a method of selecting a device for placement within an anatomical structure on an ultrasound image feed, in accordance with at least one embodiment of the present invention;

FIG. 4A is an image of a display interface with an ultrasound image feed showing an anatomical structure, in accordance with at least one embodiment of the present invention;

FIG. 4B is the display interface of FIG. 4A following deployment of the AI model for identifying and predicting one or more dimensions of the anatomical structure, in accordance with at least one embodiment of the present invention;

FIG. 4C is the display interface of FIG. 4A following deployment of the AI model for identifying and predicting one or more dimensions of the anatomical structure, in accordance with at least one embodiment of the present invention;

FIG. 4D is a graphical representation of automatically selecting a device from a plurality of devices for the anatomical structure identified within the ultrasound image feed shown in FIG. 4A in accordance with at least one embodiment of the present invention;

FIG. 4E is the display interface of FIG. 4A following deployment of the AI model for identifying and predicting one or more dimensions of the anatomical structure, in accordance with at least one embodiment of the present invention;

FIG. 5A is an image of a display interface with an ultrasound image feed showing an anatomical structure, in accordance with at least one embodiment of the present invention;

FIG. 5B is the display interface of FIG. 5A following deployment of the AI model for identifying and predicting one or more dimensions of the anatomical structure, in accordance with at least one embodiment of the present invention;

FIG. 5C is the display interface of FIG. 5A following deployment of the AI model for identifying and predicting one or more dimensions of the anatomical structure, in accordance with at least one embodiment of the present invention;

FIG. 6A is an image of a display interface with an ultrasound image feed showing an anatomical structure, in accordance with at least one embodiment of the present invention;

FIG. 6B is the display interface of FIG. 6A following deployment of the AI model for identifying and predicting one or more dimensions of the anatomical structure, in accordance with at least one embodiment of the present invention;

FIG. 6C is the display interface of FIG. 6A following deployment of the AI model for identifying and predicting one or more dimensions of the anatomical structure, in accordance with at least one embodiment of the present invention;

FIG. 6D shows a graphical representation of automatically selecting a device from a plurality of devices for the anatomical structure identified within the ultrasound image feed shown in FIG. 6A in accordance with at least one embodiment of the present invention;

FIG. 6E is the display interface of FIG. 6A following deployment of the AI model for identifying and predicting one or more dimensions of the anatomical structure, in accordance with at least one embodiment of the present invention;

FIG. 7A is an image of a display interface with an ultrasound image feed showing an anatomical structure, in accordance with at least one embodiment of the present invention;

FIG. 7B is the display interface of FIG. 7A following deployment of the AI model for identifying and predicting one or more dimensions of the anatomical structure, in accordance with at least one embodiment of the present invention;

FIG. 7C is the display interface of FIG. 7A following deployment of the AI model for identifying and predicting one or more dimensions of the anatomical structure, in accordance with at least one embodiment of the present invention;

FIG. 7D shows a graphical representation of automatically selecting a device from a plurality of devices for the anatomical structure identified within the ultrasound image feed shown in FIG. 7A in accordance with at least one embodiment of the present invention;

FIG. 7E is the display interface of FIG. 7A following deployment of the AI model for identifying and predicting one or more dimensions of the anatomical structure, in accordance with at least one embodiment of the present invention;

FIG. 8A is an image of a display interface with an ultrasound image feed showing an anatomical structure, in accordance with at least one embodiment of the present invention;

FIG. 8B is the display interface of FIG. 8A following deployment of the AI model for identifying and predicting one or more dimensions of the anatomical structure, in accordance with at least one embodiment of the present invention;

FIG. 8C shows a graphical representation of automatically selecting a device from a plurality of devices for the anatomical structure identified within the ultrasound image feed shown in FIG. 8A in accordance with at least one embodiment of the present invention;

FIG. 8D is the display interface of FIG. 8A following deployment of the AI model for identifying and predicting one or more dimensions of the anatomical structure, in accordance with at least one embodiment of the present invention;

FIG. 9 is a flowchart diagram showing steps of another example method of selecting a device for placement within an anatomical structure on an ultrasound image feed, in accordance with at least one embodiment of the present invention;

FIG. 10 is a flowchart diagram showing steps of another example method of selecting a device for placement within an anatomical structure on an ultrasound image feed, in accordance with at least one embodiment of the present invention;

FIG. 11 is a schematic diagram of the training and deployment of an AI model, according to an embodiment of the present invention; and

FIG. 12 is flowchart diagram of the steps for training the AI model, according to an embodiment of the present invention;

Unless otherwise specifically noted, articles depicted in the drawings are not necessarily drawn to scale.

### DETAILED DESCRIPTION OF THE INVENTION

### A. Glossary

The term “AI model” means a mathematical or statistical model that may be generated through artificial intelligence techniques such as machine learning and/or deep learning. For example, these techniques may involve inputting labeled or classified data into a neural network (e.g., a deep neural network) algorithm for training, so as to generate a model that can make predictions or decisions on new data without being explicitly programmed to do so. Different software tools (e.g., TensorFlow™, PyTorch™, Keras™) may be used to perform machine learning processes. Within the scope of the invention, an AI model is trained to identify and predict one or more dimensions of an anatomical structure, and to automatically select the device for placement based on the dimensions. It is to be understood that the present invention is not to be limited to any one means of deploying the AI model for such detection.

The term “communications network” and “network” can include both a mobile network and data network without limiting the term's meaning, and includes the use of wireless (e.g. 2G, 3G, 4G, 5G, WiFi®, WiMAX®, Wireless USB (Universal Serial Bus), Zigbee®, Bluetooth® and satellite), and/or hard wired connections such as local, internet, ADSL (Asymmetrical Digital Subscriber Line), DSL (Digital Subscriber Line), cable modem, T1, T3, fiber-optic, dial-up modem, television cable, and may include connections to flash memory data cards and/or USB memory sticks where appropriate. A communications network could also mean dedicated connections between computing devices and electronic components, such as buses for intra-chip communications.

The term “labeling” refers to an act of labeling either a piece of training data or non-training data. For example, a user may mark a feature on an ultrasound image and identify the anatomy to which the feature corresponds. The result is a labeled piece of data, such as a labeled ultrasound image. Alternatively, and by way of example, an AI model may automatically and without user intervention label one or more segmented features, within an ultrasound image.

The term “module” can refer to any component in this invention and to any or all of the features of the invention without limitation. A module may be a software, firmware or hardware module (or part thereof), and may be located or operated within, for example, in the ultrasound scanner, a display device or a server.

The term “multi-purpose electronic device” or “display device” or “computing device” or “off-the-shelf display computing device” is intended to have broad meaning and includes devices with a processor communicatively operable with a screen interface, for example, such as, laptop computer, a tablet computer, a desktop computer, a smart phone, a smart watch, spectacles with a built-in display, a television, a bespoke display or any other display device that is capable of being communicably connected to an ultrasound scanner. Such a device may be communicatively operable with an ultrasound scanner and/or a cloud-based server (for example via one or more communications networks).

The term “operator” (or “user”) may (without limitation) refer to the person that is operating an ultrasound scanner (for example, a clinician, medical personnel, a sonographer trainer, a student, a vet, a sonographer/ultrasonographer and/or ultrasound technician). This list is non-exhaustive.

The term “processor” can refer to any electronic circuit or group of circuits that perform calculations, and may include, for example, single or multicore processors, multiple processors, an ASIC (Application Specific Integrated Circuit), and dedicated circuits implemented, for example, on a reconfigurable device such as an FPGA (Field Programmable Gate Array). A processor may perform the steps in the flowcharts and sequence diagrams, whether they are explicitly described as being executed by the processor or whether the execution thereby is implicit due to the steps being described as performed by the system, a device, code or a module. The processor, if comprised of multiple processors, may be located together or geographically separate from each other. The term includes virtual processors and machine instances as in cloud computing or local virtualization, which are ultimately grounded in physical processors.

The term “scan convert”, “scan conversion”, or any of its grammatical forms refers to the construction of an ultrasound media, such as a still image or a video, from lines of ultrasound scan data representing echoes of ultrasound signals. Scan conversion may involve converting beams and/or vectors of acoustic scan data which are in polar (R-theta) coordinates to cartesian (X-Y) coordinates.

The term “system” when used herein, and not otherwise qualified, refers to a system for selection a device for placement within the anatomical structure on an ultrasound image frame. In various embodiments, the system may include an ultrasound scanner and a multi-purpose electronic device/display device; and/or an ultrasound scanner, multi-purpose electronic device/display device and a server. The system may include one or more applications operating on a multi-purpose electronic device/display device to which the ultrasound scanner is communicatively connected.

The term “ultrasound image frame” (or “image frame” or “ultrasound frame”) refers to a frame of either pre-scan data or post-scan conversion data that is suitable for rendering an ultrasound image on a screen or other display device.

The term “ultrasound transducer” (or “probe” or “ultrasound probe” or “transducer” or “ultrasound scanner” or “scanner”) refers to a wide variety of transducer types including but not limited to linear transducer, curved transducers, curvilinear transducers, convex transducers, microconvex transducers, and endocavity probes. In operation, an ultrasound scanner is often communicatively connected to a multi-purpose electronic device/display device to direct operations of the ultrasound scanner, optionally through one or more applications on the multi-purpose electronic device/display device (for example, via the Clarius™ App).

The term “workflow application” or “application” (for example, via the Clarius™ App) or “workflow” refers to a software tool that automates the tasks involved in the device selection process including, but not limited to the following method steps: i) displaying, on a screen communicatively connected to the ultrasound scanner, the ultrasound image feed comprising the anatomical structure; ii) deploying an AI model to execute on a computing device communicatively connected to the ultrasound scanner, wherein the AI model is trained so that when the AI model is deployed, the computing device identifies and predicts one or more dimensions of the anatomical structure; iii) acquiring, at the computing device, a new ultrasound image during ultrasound scanning; iv) processing, using the AI model, the new ultrasound image to identify and predict the one or more dimensions of the anatomical structure; and v) automatically selecting a device from the plurality of devices for placement therein based on the one or more dimensions. It is to be understood that a workflow application and/or software tool may facilitate some or all of the method tasks as described herein. More specifically in some aspects of the invention, one or more dimension measurements only require that the workflow tool be activated once, where the workflow enables: i) activation of an AI model to identify and segment an anatomical structure; and ii) automatic determination and calculation of one or more dimensions of the anatomical structure based upon the AI model generated segmented anatomical structure; and iii) capture of one or more dimensions (the “dimensions”) and employment of the dimensions in the selection of a device for placement.

### B. Exemplary Embodiments

In a first broad aspect of the present disclosure, there are provided ultrasound systems, ultrasound-based methods, tools and workflows for selecting a device for placement within an anatomical structure on an ultrasound image feed that is acquired from an ultrasound scanner.

In another aspect of the present disclosure, there is provided a method of selecting from a plurality of devices for placement within an anatomical structure on an ultrasound image feed that is acquired from an ultrasound scanner, the method comprising: displaying, on a screen communicatively connected to the ultrasound scanner, the ultrasound image feed comprising the anatomical structure; deploying an AI model to execute on a computing device communicatively connected to the ultrasound scanner, wherein the AI model is trained so that when the AI model is deployed, the computing device identifies and predicts one or more dimensions of the anatomical structure; acquiring, at the computing device, a new ultrasound image during ultrasound scanning; processing, using the AI model, the new ultrasound image to identify and predict the one or more dimensions of the anatomical structure; and automatically selecting a device from the plurality of devices for placement therein based on the one or more dimensions.

In another aspect, the method further comprises: applying the AI model to segment boundaries of the anatomical structure in the new ultrasound image, and generating a segmented anatomical structure for display on the screen.

In another aspect, the method of claim 1, wherein the screen is within a multi-purpose electronic device which is communicatively coupled with the ultrasound scanner and an additional step of indicating the device, which is automatically selected, is via at least one of a visual signal on the display or an audio signal.

In another aspect, the method of claim 1 further comprises: applying the AI model to identify a diameter of the anatomical structure; and applying the AI model to automatically select the device for placement based on the diameter.

In another aspect, wherein more than one device is selected by the AI model based on the diameter of the anatomical structure, and an additional step comprises the AI model selecting a preferred device, of the more than one device, based upon a clinical application.

In another aspect, the method further comprises: applying the AI model to select the size of the device from a plurality of devices based on at least one of i) characteristics of the anatomical structure; ii) characteristics of a patient; iii) a clinical application; iv) best practices for device placement; and v) historical records. The AI model can i) identify two devices of two different sizes from the plurality of devices, and ii) select a smaller size from the two different sizes.

In another aspect, the method further comprises: identifying a standardized size for the device based on the one or more dimensions of the anatomical structure; and selecting the size of the device that corresponds to the standardized size.

In another aspect, the method further comprises: applying the AI model to select the size of the endotracheal tube from two different sized endotracheal tubes based on at least one of: i) purpose of endotracheal tube placement; ii) characteristics of the trachea; iii) characteristics of a patient; iv) a clinical application; v) best practices for endotracheal tube placement; and vi) historical records.

In another aspect, the one or more dimensions is selected from the group consisting of a diameter of the anatomical structure, a length of the anatomical structure, a width of the anatomical structure, circumference of the anatomical structure, an area of the anatomical structure, and a height of the anatomical structure.

In another aspect, the device is selected from the group consisting of a catheter, endotracheal tube and an implant. The device can be a catheter, the one of more dimensions is an internal diameter of the anatomical structure and a size of the catheter is automatically selected by the AI model, based upon a measurement gauge of an external diameter of the catheter, as compared to a best fit of the internal diameter of the anatomical structure. The device can be endotracheal tube, the one of more dimensions is an internal diameter of a trachea and a size of the endotracheal tube is automatically selected by the AI model, based upon a measurement gauge of an external diameter of the endotracheal tube. The implant can be selected from the group consisting of spinal implants, orthopedic implants, neurological implants, vascular implants, and cardiac implants.

In another aspect, the AI model is trained with a plurality of training ultrasound images comprising labelled segmented boundaries of the anatomical structure, in plurality of views, which are, one of: i) generated by one of a manual or semi automatic means; or ii) tagged from an identifier menu by one of a manual, semi automatic means or fully automatic means.

In another aspect, the method comprising training the AI model with one or more of the following: i) supervised learning; ii) unsupervised learning; iii) previously labelled ultrasound image datasets; and iv) cloud stored data.

In another aspect of the present disclosure, there is provided a system for selecting a plurality of devices for placement within an anatomical structure on an ultrasound image frame, the system comprising: an ultrasound scanner configured to acquire the ultrasound image frame of the anatomical structure; a display device communicatively connected to the ultrasound scanner, the display device comprising a screen configured to display the ultrasound image frame; and a computing device communicatively connected to the ultrasound scanner and configured to: process the ultrasound image frame against an AI model trained to identify and predict one or more dimensions of the anatomical structure; and automatically select a device from the plurality of devices for placement therein based on the one or more dimensions.

In another aspect of the present disclosure, there is provided a computer-readable media storing computer-readable instructions, which, when executed by a processor cause the processor to: display, on a screen communicatively connected to the ultrasound scanner, the ultrasound image feed comprising the anatomical structure; deploy an AI model to execute on a computing device communicatively connected to the ultrasound scanner, wherein the AI model is trained so that when the AI model is deployed, the computing device identifies and predicts one or more dimensions of the anatomical structure; acquire, at the computing device, a new ultrasound image during ultrasound scanning; process, using the AI model, the new ultrasound image to identify and predict the one or more dimensions of the anatomical structure; and automatically select a device from the plurality of devices for placement therein based on the one or more dimensions.

In present invention, an artificial intelligence (AI) model is trained on a plurality of ultrasound images of anatomy/anatomical features, for the purpose of feature classification and/or boundary segmentation as described further below. The AI model can be trained with one or more supervised learning datasets, unsupervised learning datasets, previously labelled ultrasound image datasets, and/or cloud stored datasets. These images enable the AI model to be trained so that when the AI model is deployed, a computing device communicably connected to an ultrasound scanner, either classifies features, in whole or part or segments boundaries of features, in whole or part, either way thereafter identifying and predicting one or more dimensions of an anatomical structure. As such, the present invention further provides, in another aspect, such a trained and deployable AI model.

The AI model can be trained with a plurality of training ultrasound images that includes labelled segmented boundaries of the anatomical structures, in plurality of views. The segmented boundaries of the anatomical structures can be generated by one of a manual or semi-automatic means, and/or tagged from an identifier menu by one of a manual, semi-automatic means or fully automatic means.

There are various methods which may be employed in AI-based segmentation of ultrasound images, and the present invention is not intended to be limited to any one of these methods. Image segmentation refers to the detection of boundaries of features and structures, such as, but not limited to organs, vessels, different types of tissue in ultrasound images. In an embodiment of the present invention, a method deploys a trained AI model to perform intelligent automated recognition of segmentation tasks and intelligent automated selection and application of segmentation algorithms. This allows the AI model to be applied to intelligently perform various different segmentation tasks, including segmentation of the anatomical structure of interest. The AI model can intelligently select one or a combination of segmentation algorithms from a plurality of segmentation algorithms to perform appropriate segmentation for various features and anatomical structures. For example, the algorithms may be a threshold-based segmentation algorithm, an edge-based segmentation algorithm, a region-based segmentation algorithm, a clustering-based segmentation algorithm, or the like, or a combination thereof.

In some embodiments of the invention, segmentation algorithms may be stored in a segmentation algorithm database which may comprise a plurality of deep learning-based ultrasound image segmentation methods, each of which may include a respective trained deep neural network architecture for performing ultrasound image segmentation. For example, the segmentation algorithms can include the deep learning based segmentation algorithms described below, including segmentation using a deep neural network (DNN) that integrates shape priors through joint training, non-rigid shape segmentation method using deep reinforcement learning, segmentation using deep learning based partial inference modeling under domain shift, segmentation using a deep-image-to-image network and multi-scale probability maps, and active shape model based segmentation using a recurrent neural network (RNN). The segmentation algorithm database may include other deep learning-based segmentation algorithms as well, such as marginal space deep learning (MSDL) and marginal space deep regression (MSDR) segmentation methods. It is also possible that a segmentation algorithm database may also store various other non-deep learning-based segmentation algorithms, including but not limited to machine-learning based segmentation methods (e.g., marginal space learning (MSL) based segmentation), graph cuts segmentation methods, region-growing based segmentation methods, and atlas-based segmentation methods.

A segmentation algorithm database may store multiple versions of each segmentation algorithm corresponding to different target anatomical features and structures. For deep learning-based segmentation algorithms, each version corresponding to a specific target anatomical structure may include a respective trained deep network architecture with parameters (weights) learned for segmentation of that target anatomical structure. For a particular anatomical structure, a segmentation algorithm database can also store multiple versions corresponding to different imaging domains and/or image quality levels. For example, different deep learning architectures can be trained and stored using images with different signal-to-noise ratios (SNRs). Accordingly, when a master segmentation artificial agent selects one or more segmentation algorithms from the those stored in a segmentation algorithm database, the master segmentation artificial agent may select not only the type of segmentation algorithm to apply, but the specific versions of segmentation algorithms that are best for performing the current segmentation task.

In some embodiments, the ultrasound frames of a new ultrasound image, imaged in ultrasound imaging data may be processed against an AI model on a per pixel basis, and thus the segmentation of boundaries of features, in whole or part, on the new ultrasound image, thereby creating one segmented boundary feature or two or more segmented boundary features, imaged in new ultrasound imaging data, may be generated on a per pixel basis. When deployed, an output of the AI model for a first pixel of the new ultrasound imaging data may be used to corroborate the output of the AI model for a second pixel of the new ultrasound imaging data adjacent or within the proximity to the first pixel.

Alternatively, the ultrasound frames of new ultrasound images, imaged in ultrasound imaging data may be processed against an AI model on a line/sample basis, and thus the segmentation of boundaries of the feature or features, in whole or part, on the new ultrasound image, thereby creating at least one or two or more segmented boundary features, imaged in new ultrasound imaging data, may be generated on a line/sample basis.

Image segmentation algorithms may automatically identify anatomical structures in ultrasound images. Currently, the dimensions of anatomical structures are gauged by medical professionals based on established age and height-based formulas. Unfortunately, there is often a significant variation in size and shape of the anatomical structure such that the correlation between age, height, weight, body surface area and anatomical structure shape or size is poor, and so, the established formulas are often accurate.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, certain steps, signals, protocols, software, hardware, networking infrastructure, circuits, structures, techniques, well-known methods, procedures and components have not been described or shown in detail in order not to obscure the embodiments generally described herein.

Furthermore, this description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

The system of the present invention uses a transducer (e.g., a piezoelectric or capacitive device operable to convert between acoustic and electrical energy) to scan a planar region or a volume of an anatomical structure. Electrical and/or mechanical steering allows transmission and reception along different scan lines wherein any scan pattern may be used. Ultrasound data representing a plane or volume is provided in response to the scanning. The ultrasound data is beamformed, detected, and/or scan converted. The ultrasound data may be in any format, such as polar coordinate, Cartesian coordinate, a three-dimensional grid, two-dimensional planes in Cartesian coordinate with polar coordinate spacing between planes, or other format. The ultrasound data is data which represents an anatomical structure sought to be assessed and reviewed by a sonographer.

A user input device may comprise one or more of a touchscreen, a keyboard, a mouse, a trackpad, a motion sensing camera, or other device configured to enable a user to interact with and manipulate data within an image processing system. In one example, user input device may enable a user to make a selection of an ultrasound image to use in training an AI model, or for further processing using a trained AI model. A display device may include one or more display devices utilizing virtually any type of technology. In some embodiments, display device may be part of a multi-purpose display device or may comprise a computer monitor, and in both cases, may display ultrasound images. A display device may be combined with processor, non-transitory memory, and/or user input device in a shared electronic device, or there may be peripheral display devices which may comprise a monitor, touchscreen, projector, or other display device known in the art, which may enable a user to view ultrasound images produced by an ultrasound imaging system, and/or interact with various data stored in non-transitory memory.

In various embodiments, a multi-purpose electronic devices/display devices may be, for example, a laptop computer, a tablet computer, a desktop computer, a smart phone, a smart watch, spectacles with a built-in display, a television, a bespoke display or any other display device that is capable of being communicably connected to an ultrasound probe.

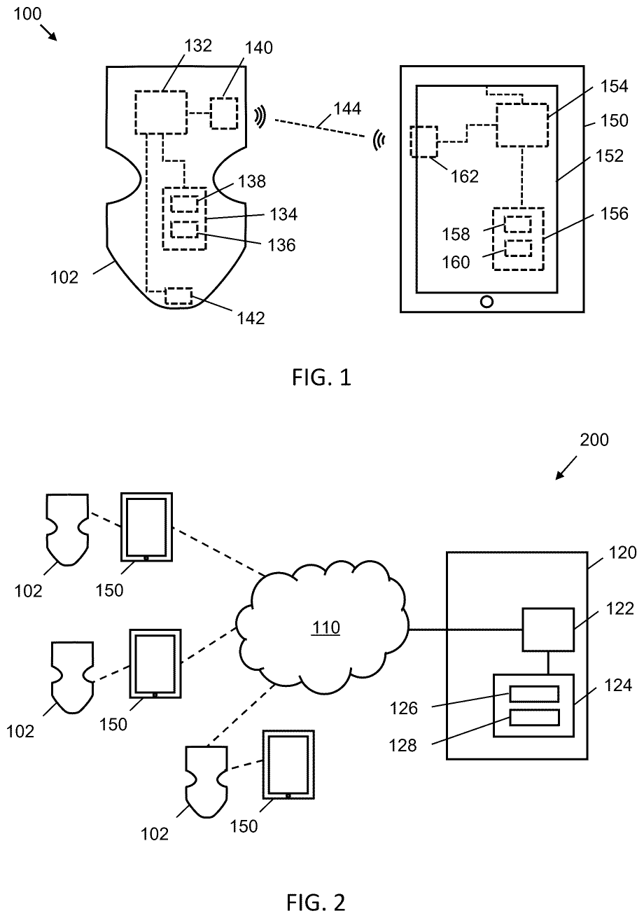

Reference will now be made to FIGS. 1 and 2. In FIG. 1, there is shown an exemplary system 100 for selecting a device for placement within an anatomical structure on an ultrasound image frame. The system 100 includes an ultrasound scanner 102 with a processor 132, which is connected to a non-transitory computer readable memory 134 storing computer readable instructions 136, which, when executed by the processor 132, may cause the scanner 102 to provide one or more of the functions of the system 100. Such functions may be, for example, the acquisition of ultrasound data, the processing of ultrasound data, the scan conversion of ultrasound data, the transmission of ultrasound data or ultrasound frames to a display device 150, the detection of operator inputs to the ultrasound scanner 102, and/or the switching of the settings of the ultrasound scanner 102.

Also stored in the computer readable memory 134 may be computer readable data 138, which may be used by the processor 132 in conjunction with the computer readable instructions 136 to provide the functions of the system 100. Computer readable data 138 may include, for example, configuration settings for the scanner 102, such as presets that instruct the processor 132 how to collect and process the ultrasound data for a plurality of regions of interest (ROIs) and how to acquire a series of ultrasound frames. The scanner 102 may include an ultrasonic transducer 142 that transmits and receives ultrasound energy in order to acquire ultrasound frames. The scanner 102 may include a communications module 140 connected to the processor 132. In the illustrated example, the communications module 140 may wirelessly transmit signals to and receive signals from the display device 150 along wireless communication link 144. The protocol used for communications between the scanner 102 and the display device 150 may be WiFi™ or Bluetooth™, for example, or any other suitable two-way radio communications protocol. In some embodiments, the scanner 102 may operate as a WiFi™ hotspot, for example. Communication link 144 may use any suitable wireless communications network connection. In some embodiments, the communication link 144 between the scanner 102 and the display device 150 may be wired. For example, the scanner 102 may be attached to a cord that may be pluggable into a physical port of the display device 150.

The display device 150 can include any multi-purpose electronic devices that can host a screen 152, and may include a processor 154, which may be connected to a non-transitory computer readable memory 156 storing computer readable instructions 158, which, when executed by the processor 154, cause the display device 150 to provide one or more of the functions of the system 100. Such functions may be, for example, the receiving of ultrasound data that may or may not be pre-processed; scan conversion of received ultrasound data into an ultrasound image; processing of ultrasound data in image data frames; the display of a user interface; the control of a probe and the display of an ultrasound image on the screen to identify and predict one or more dimensions of the anatomical structure.

In various embodiments, the display device 150 may be, for example, a laptop computer, a tablet computer, a desktop computer, a smart phone, a smart watch, spectacles with a built-in display, a television, a bespoke display or any other display device that is capable of being communicably connected to the scanner 102. The screen 152 may comprise a touch-sensitive display (e.g., touchscreen) that can detect a presence of a touch from the operator on screen 152 and can also identify a location of the touch in screen 152. The touch may be applied by, for example, at least one of an individual's hand, glove, stylus, or the like. As such, the touch-sensitive display may be used for example to toggle text or to provide other inputs regarding the measurements and calculated volume. The screen 152 and/or any other user interface may also communicate audibly. The display device 150 is configured to present information to the operator during or after the imaging or data acquiring session. The information presented may include ultrasound images (e.g., one or more 2D frames), graphical elements, measurement graphics of the displayed images, user-selectable elements, user settings, and other information (e.g., administrative information, personal information of the patient, and the like).

Also stored in the computer readable memory 156 may be computer readable data 160, which may be used by the processor 154 in conjunction with the computer readable instructions 158 to provide the functions of the system 100. Computer readable data 160 may include, for example, settings for the scanner 102, such as presets for acquiring ultrasound data; settings for a user interface displayed on the screen 152; and/or data for one or more AI models within the scope of the invention. Settings may also include any other data that is specific to the way that the scanner 102 operates or that the display device 150 operates. It can therefore be understood that the computer readable instructions and data used for controlling the system 100 may be located either in the computer readable memory 134 of the scanner 102, the computer readable memory 156 of the display device 150, and/or both the computer readable memories 134, 156.

The display device 150 may also include a communications module 162 connected to the processor 154 for facilitating communication with the scanner 102. In the illustrated example, the communications module 162 wirelessly transmits signals to and receives signals from the scanner 102 on wireless communication link 144. However, as noted, in some embodiments, the connection between scanner 102 and display device 150 may be wired.

Such a screen may comprise a touch-sensitive display (e.g., touchscreen) that can detect a presence of a touch from the operator on screen and can also identify a location of the touch in screen. The touch may be applied by, for example, at least one of an individual's hand, glove, stylus, or the like. As such, the touch-sensitive display may be used to receive an input, for example, indicating the presence or absence of text or annotations on an image. The screen and/or any other user interface may also communicate audibly. The display device 150 may be configured to present information to the operator during or after the imaging or data acquiring session. The information presented may include ultrasound images (e.g., one or more 2D frames), graphical elements, measurement graphics of the displayed images, user-selectable elements, user settings, and other information (e.g., administrative information, personal information of the patient, and the like).

Referring to FIG. 2, a system 200 is shown in which there are multiple similar or different scanners 102 connected to their corresponding display devices 150 and either connected directly, or indirectly via the display devices, to a communications network 110, such as the internet.

The scanners 102 may be connected via the communications network 110 to a server 120. The server 120 may include a processor 122, which may be connected to a non-transitory computer readable memory 124 storing computer readable instructions 126, which, when executed by the processor 122, cause the server 120 to provide one or more of the functions of the system 100. Such functions may be, for example, the receiving of ultrasound frames, the processing of ultrasound data in ultrasound frames, the control of the scanners 102, the processing of using the AI model on new ultrasound images to identify and predict the one or more dimensions of the anatomical structure.

Also stored in the computer readable memory 124 may be computer readable data 128, which may be used by the processor 122 in conjunction with the computer readable instructions 126 to provide the functions of the system 100. Computer readable data 128 may include, for example, settings for the scanners 102 such as preset parameters for acquiring ultrasound data, settings for user interfaces displayed on the display devices 150, and data for one or more AI models. Settings may also include any other data that is specific to the way that the scanners 102 operate or that the display devices 150 operate.

It can therefore be understood that the computer readable instructions and data used for controlling the system 100 may be located either in the computer readable memory of the scanners 102, the computer readable memory of the display devices 150, the computer readable memory 124 of the server 120, or any combination of the foregoing locations.

As noted above, even though the scanners 102 may be different, each ultrasound frame acquired may be used by the AI model for training purposes. Likewise, ultrasound frames acquired by the individual scanners 102 may all be processed against the AI model for reinforcement of the AI model. In some embodiments, the AI models present in the display devices 150 may be updated from time to time from an AI model present in the server 120, where the AI model present in the server 120 is continually trained using ultrasound frames of additional data acquired by multiple scanners 102.

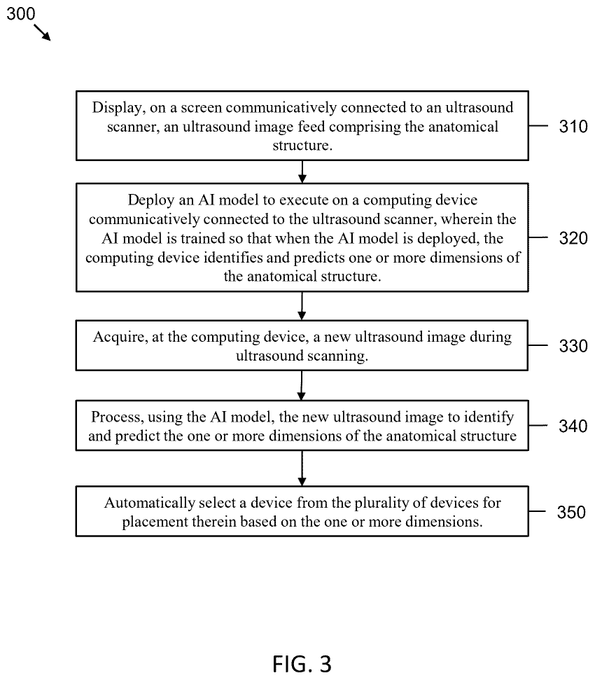

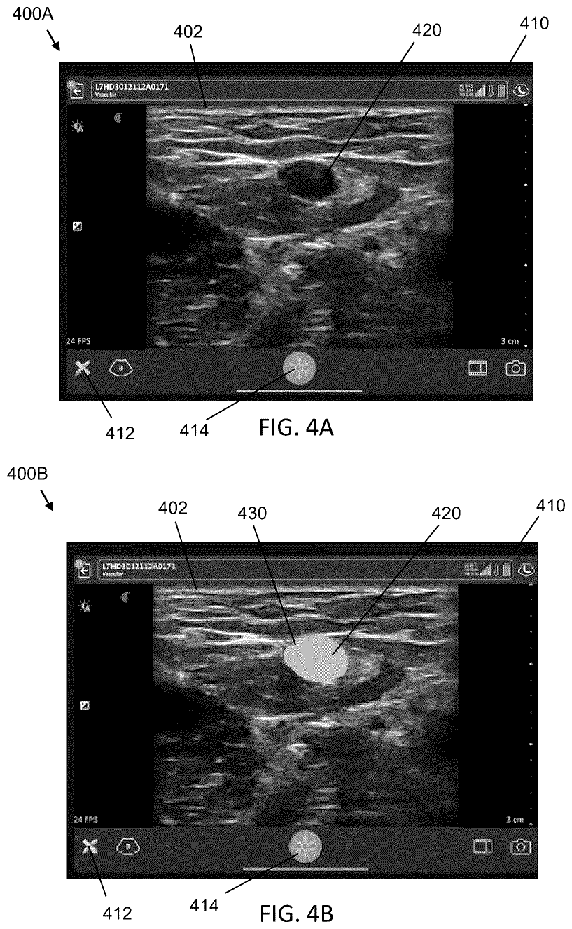

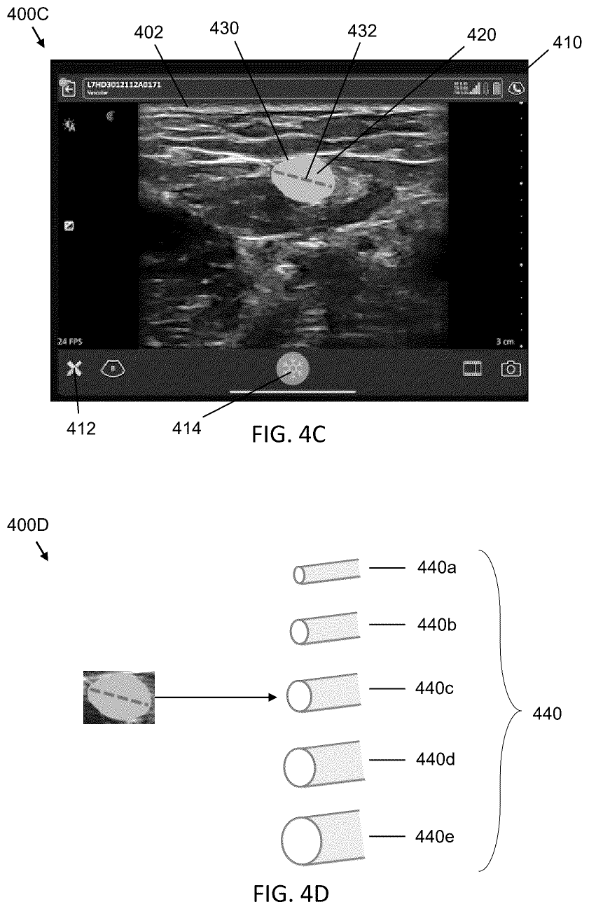

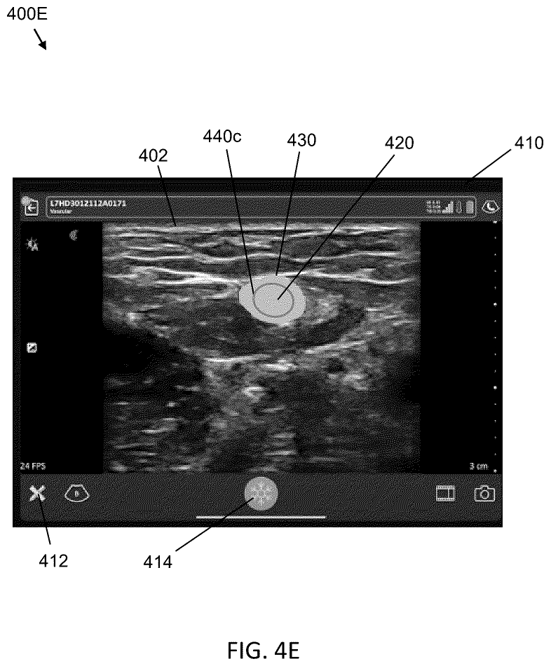

Referring to FIG. 3, there is shown a flowchart diagram of a method, generally indicated at 300, of selecting from a plurality of devices for placement within an anatomical structure on an ultrasound image feed acquired from the ultrasound scanner 102. Reference will also be made to FIGS. 4A to 4E. For ease of exposition, the example method described with reference to FIGS. 3 to 4E is with respect to system 200 of FIG. 2. In some embodiments, the one or more steps of the method 300 can be provided by the server 120, the ultrasound scanner 102 and/or the display device 150. In some embodiments, the system 100 can perform the method 300 without the server 120.

At 310, the system 200 displays, on a screen 152 communicatively connected to the ultrasound scanner 102 the ultrasound image feed comprising the anatomical structure.

The present invention provides a means of selecting a device, from a plurality of devices, for placement within an anatomical structure on an ultrasound image feed, using a trained AI model which identifies and predicts one or more dimensions of the anatomical structure such one or more dimensions being employed to automatically select a device from the plurality of devices. It is to be understood that the device, in the context of the invention, is to be accorded a wide interpretation and includes, but is not limited to a catheter, an endotracheal tube and an implant. Example catheters can include those for arterial and/or venous peripheral line placement and central line placement and dialysis. Example implants can include stents (for example, arterial stents and bile duct stents), spinal implants, orthopedic implants (for example, joint prostheses), neurological implants, vascular implants, urological implants, and cardiac implants (for example replacement cardiac valves). An anatomical structure, within the context of the invention comprises one or more dimensions which are identified by the deployed and trained AI model, for the purpose of sizing and selecting a device (i.e. the one or more dimensions guiding device size selection, at least in part) and includes, but is not limited to, arterial and venous vessels, a trachea, ducts (including a bile duct, a hepatic duct, and a pancreatic duct), a urethra, cardiac features (such as, for example, cardiac valves) and MSK features (such as, for example joint prostheses).

In vascular access practices, the internal vessel size is considered important to avoid catheter related thrombosis and catheter dysfunction, and a catheter to vessel ratio (CVR), or the dwelling space or area consumed or occupied by an intravascular device inserted and positioned within a venous or arterial blood vessel. For example, in peripherally inserted central catheters (PICCs), the risk of deep vein thrombosis for improperly sized catheters is significant. It has been found that more appropriately sized and smaller-gauge PICCs occupy less cross-sectional venous area thus allowing greater blood flow around the catheter, substantially reducing this risk of DVT. Fitting a catheter within a vessel correctly, based on patient size, health, vessel size etc., is thus of paramount importance. The present invention enables a user/clinician to automate this sizing and device selection decision through the deployment of a specifically trained AI model and associated workflow.

Recent best practices recommend that the CVR can increase from 33 to 45% of a vessel's diameter. For example, 33% would mean that when a vessel was measured, one-third (⅓) of the vessel's diameter should be consumed by catheter and two-thirds (⅔) should remain unobstructed to allow adequate blood flow dynamics around the device.

Within the range of available vascular accesses, different measurement units are used for the external and internal diameter of a catheter. For example, a short catheter is categorized by a diameter measured in Gauges (G), measuring the internal diameter of the catheter wherein the wider the diameter, the smaller the measurement in G, and vice versa. Midlines, PICC, reservoirs, and Hickman catheters have a diameter whose measurement is expressed in French (Fr) or French scale. In this case, the measurement in Fr (measuring the external diameter of the catheter) varies in the same way as the device: a small catheter will have a small French, and vice versa. In central catheters, it is possible to observe both units of measure: the external diameter of the catheter expressed in French and the diameter of the lumens expressed in Gauges. By way of example, Table 1 below, excerpted from “Reducing catheter related thrombosis using a risk reduction tool centered on catheter to vessel ratio” (Timothy Spencer & Keegan Mahoney: J. Thrombosis DOI 10.1007/s11239-017-1569-y, Vascular Access 31st Annual Scientific Meeting September 2017), the entire contents of which are incorporated here by reference, sets out preferred CVR ranges, along with catheter diameters, as may be employed within one embodiment of the invention, wherein vessel size/diameters are measured in millimeters:

TABLE 1

| | | Vessel Size | | | | | | | | | | |
|---|---|---|---|---|---|---|---|---|---|---|---|---|
| Catheter Size | | 1 mm | 1.5 mm | 2 mm | 2.25 mm | 2.5 mm | 2.75 mm | 3 mm | 3.5 mm | 4 mm | 4.5 mm | 5 mm |
| 24G | | X | ✓ | ✓ | ✓ | ✓ | ✓ | ✓ | ✓ | ✓ | ✓ | ✓ |
| 22G | | X | ◯ | ✓ | ✓ | ✓ | ✓ | ✓ | ✓ | ✓ | ✓ | ✓ |
| 20G | | X | X | ✓ | ✓ | ✓ | ✓ | ✓ | ✓ | ✓ | ✓ | ✓ |
| 18G | | X | X | ◯ | ◯ | ✓ | ✓ | ✓ | ✓ | ✓ | ✓ | ✓ |
| 16G | | X | X | X | X | X | ◯ | ◯ | ✓ | ✓ | ✓ | ✓ |
| 1 | Fr | ✓ | ✓ | ✓ | ✓ | ✓ | ✓ | ✓ | ✓ | ✓ | ✓ | ✓ |
| 2 | Fr | ◯ | ✓ | ✓ | ✓ | ✓ | ✓ | ✓ | ✓ | ✓ | ✓ | ✓ |
| 3 | Fr | X | ◯ | ✓ | ✓ | ✓ | ✓ | ✓ | ✓ | ✓ | ✓ | ✓ |
| 4 | Fr | X | X | ◯ | ◯ | ✓ | ✓ | ✓ | ✓ | ✓ | ✓ | ✓ |
| 4.5 | Fr | X | X | X | ◯ | ◯ | ✓ | ✓ | ✓ | ✓ | ✓ | ✓ |
| 5 | Fr | X | X | X | X | ◯ | ◯ | ✓ | ✓ | ✓ | ✓ | ✓ |
| 5.5 | Fr | X | X | X | X | X | ◯ | ✓ | ✓ | ✓ | ✓ | ✓ |
| 6 | Fr | X | X | X | X | X | X | ◯ | ✓ | ✓ | ✓ | ✓ |
| 7 | Fr | X | X | X | X | X | X | X | ◯ | ✓ | ✓ | ✓ |
| 8 | Fr | X | X | X | X | X | X | X | X | ◯ | ✓ | ✓ |

*Table 1 is coded as follows: X = ≥45% ◯ = 44-3|4% ✓ = ≤33%

Endotracheal intubation is a commonly performed procedure but can lead to complications due to improper size of the endotracheal tube. Smaller diameter tubes are easier to insert and require less force to adapt to the patient's airway but are associated with higher resistance, difficulty in passing a suction catheter and increased risk of occlusion, aspiration, and kinking with insufficient ventilation. Conversely, larger tubes are associated with higher incidence of postoperative sore throat, may damage the tracheal mucosa, can cause airway edema, post-extubation stridor, subglottic stenosis due to inflated cuff. Moreover, there is great variation in size and shapes of trachea and the correlation between age, height, weight, body surface area and whether a tracheal shape or size is poor. At present, an endotracheal tube is selected according to simple age and height-based formulas, which generally predict either smaller or larger tube sizes than are clinically optimal. This selection is greatly exacerbated in selecting tubes for children. The size of an endotracheal tube is given by its internal diameter rather than outer diameter or length. The internal diameter is relevant to the safety and conduct of anaesthesia, whilst the outer diameter is relevant to airway trauma because it is this that must be accommodated by the airway. The known formula tends to recommend 7.0-mm (internal diameter) tubes for women and 8.0-mm tubes for men undergoing routine anaesthesia, although this non-patient tailored approach leads to potential airflow limitations and suboptimal tracheal seal, if tubes with too small a diameter are selected. Furthermore, the outer diameter of a standard 8.0-mm tracheal tube is greater than 10.5 mm, therefore in some patients it would not pass through the subglottic portions of the airway without significant mucosal trauma. The present invention enables a user/clinician to automate this sizing and device selection decision through the deployment of a specifically trained AI model and associated workflow.

A stent is a tiny plastic or mesh tube that may be permanently placed in an artery, other blood vessel, or duct (such as for example, a bile duct or in a urinary passage) to maintain integrity and opening. By way of example, stents can also open up arteries narrowed by peripheral arterial disease and may be used to treat an abdominal aortic aneurysm. Whereas the typical size of an abdominal aorta, for example, is 2.0 to 3.0 centimeters, an enlarged abdominal aorta is typically greater than 3.0 centimeters. A self-expanding stent's chronic outward force (COF) is dependent on the stent's design and materials, the structure of the lesion, as well as the implanted stent's selected size for a target vessel diameter. Self-expanding stents should generally be at least one size larger than the vessel or duct diameter to ensure adequate contact with the vessel or duct wall; however, the greater the size ratio, the more COF is exerted onto the vessel or duct wall, which can result in mechanical stress that may increase neointimal hyperplasia and restenosis, among other complications. As such, stent size selection is critical for good clinical outcomes. The present invention enables a user/clinician to automate this sizing and device selection decision through the deployment of a specifically trained AI model and associated workflow.

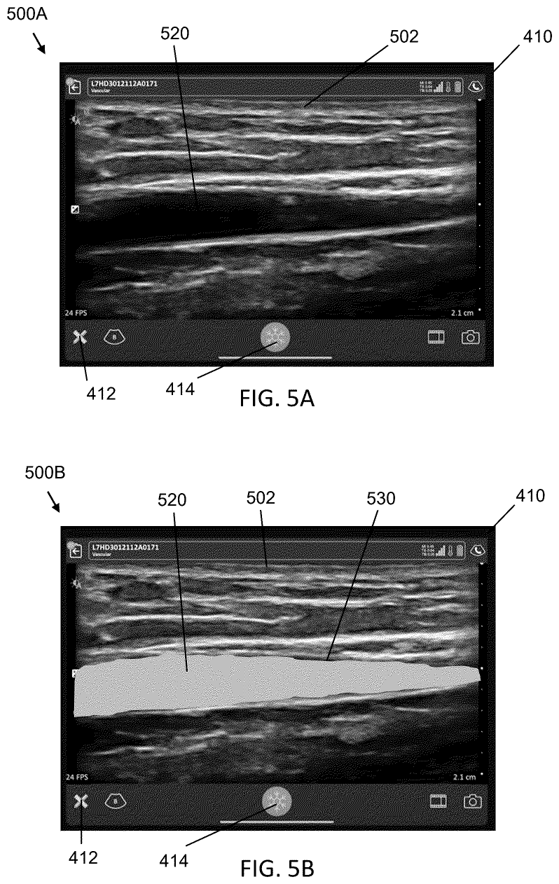

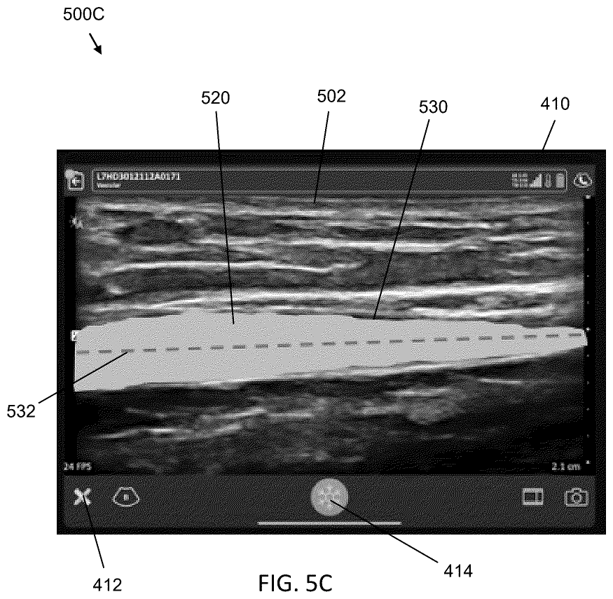

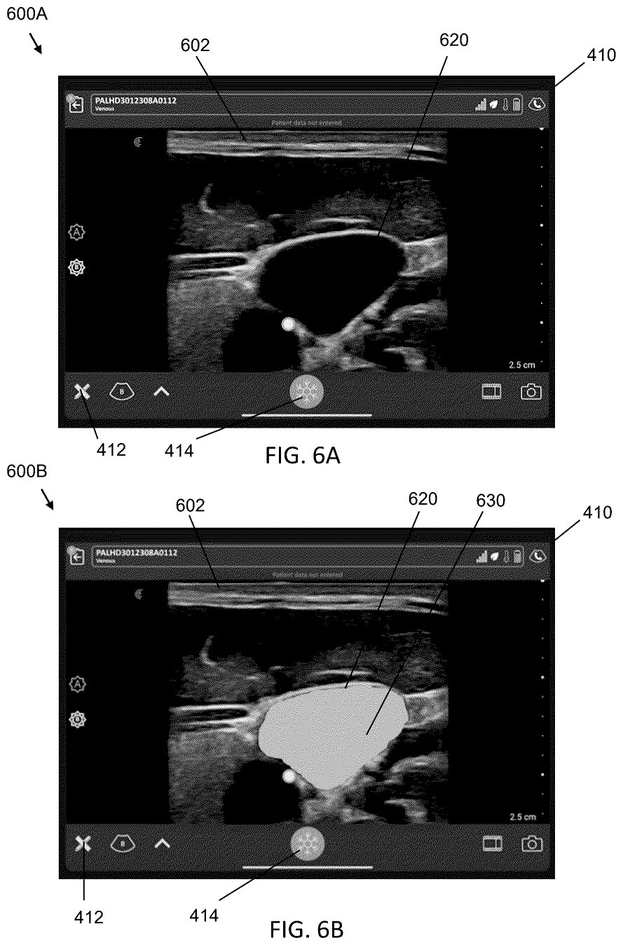

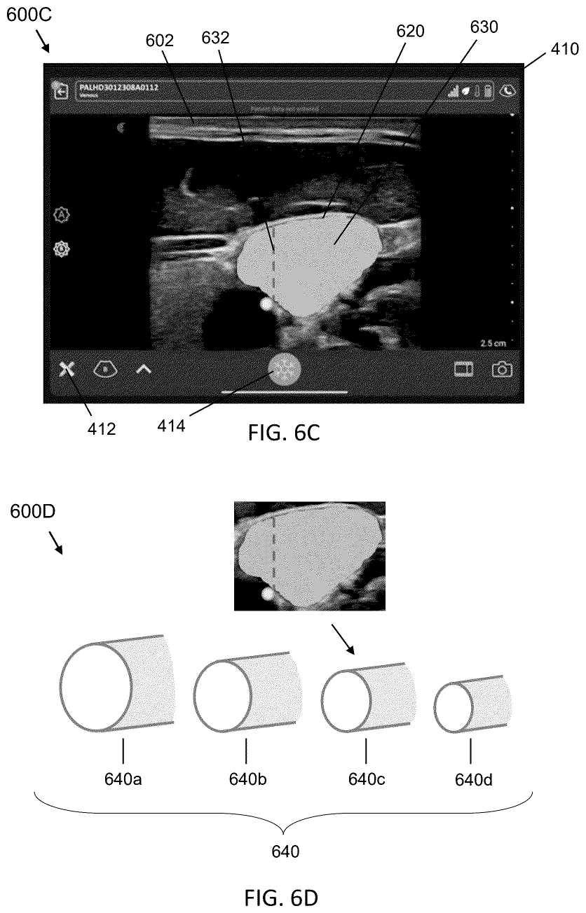

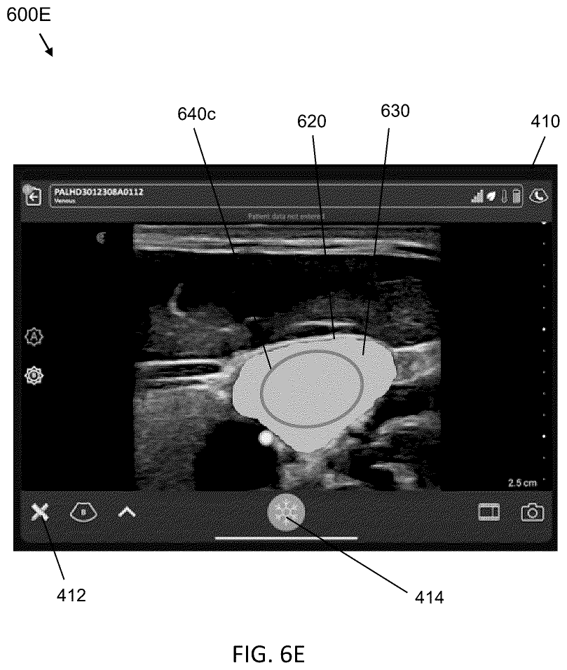

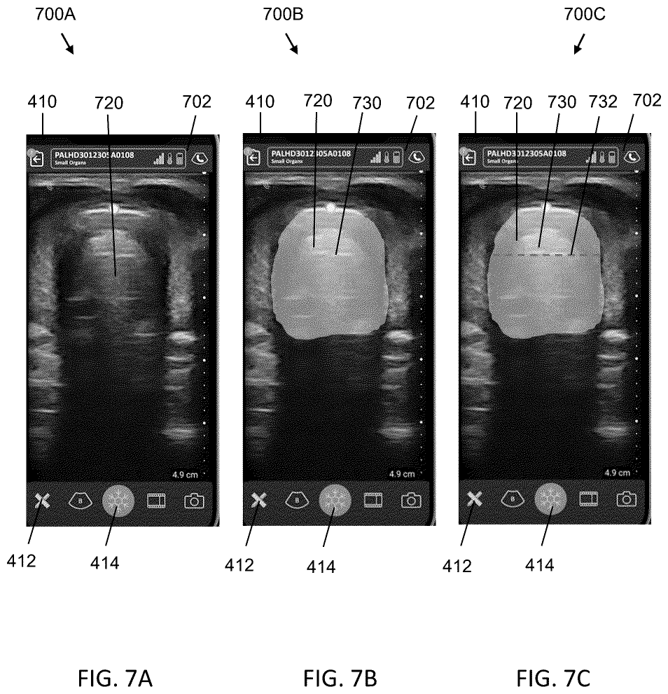

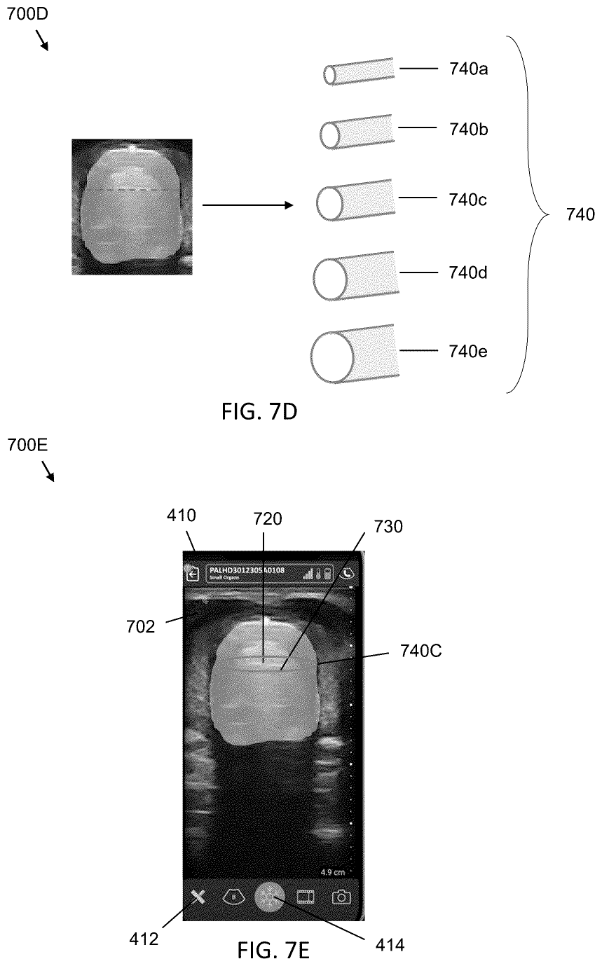

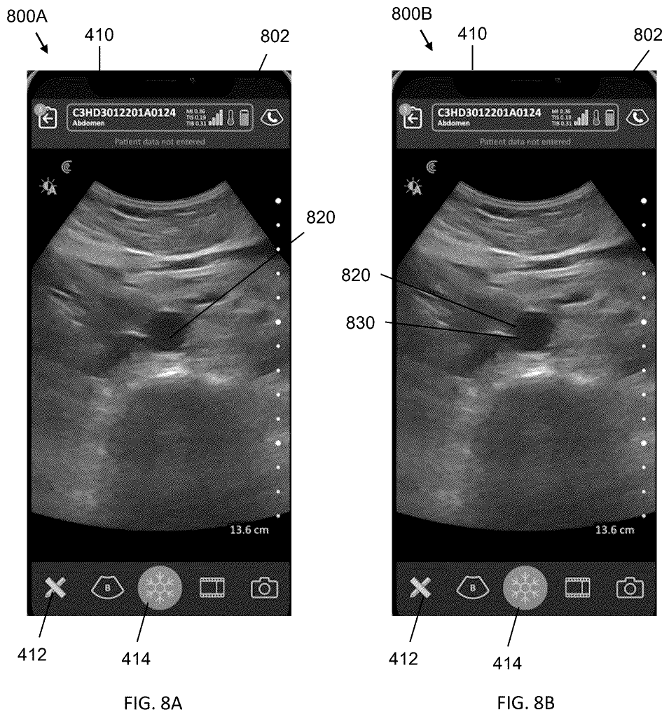

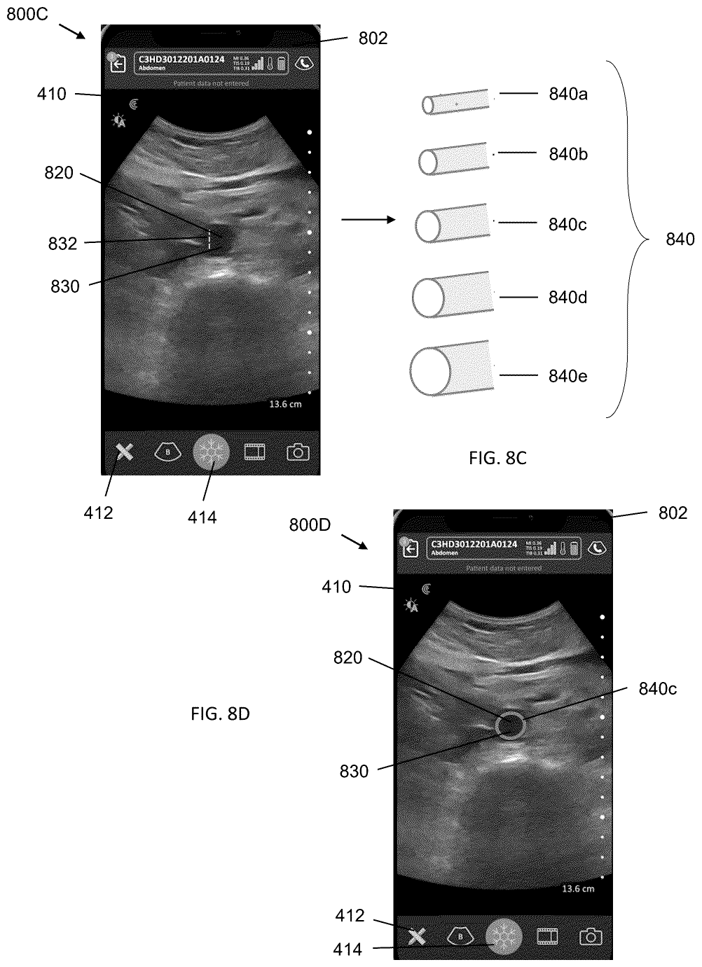

By way of example and as described further below, in FIGS. 4A-4E, an anatomical structure is a basilic vein, in transverse view, a dimension is a diameter of the basilic vein, and a device is a peripheral line catheter. In FIGS. 5A-5C, an anatomical structure is an adductor canal, in longitudinal view, and a device is a catheter. In FIGS. 6A-6E, an anatomical structure is a jugular vein, in transverse view, a dimension is a diameter of the jugular vein, and a device is a central line catheter. In FIGS. 7A-7E, an anatomical structure is a trachea, in transverse view, a dimension is a diameter of the trachea, and a device is an endotracheal tube. In FIGS. 8A-8D, an anatomical structure is an abdominal aorta, in transverse view, a dimension is a diameter of the abdominal aorta, and a device is a stent.

FIG. 4A is an image 400A of a display interface with an ultrasound image feed showing an anatomical structure 420.

In this example, the image 400A is shown within a display interface 410 of a multi-purpose electronic device, such as the display device 150 described with reference to FIGS. 1 and 2, or a separate computing device communicatively connected to the ultrasound scanner 102. In FIG. 4A, the display interface 410 shows acquisition of a B-mode image of a region of interest that includes a basilic vein 420. Basilic veins are often an anatomical structure 420 for peripheral venous catheter placement (PICC).

At 320, the system 200 deploys an AI model to execute on the computing device 150 communicatively connected to the ultrasound scanner 102.

As described, the AI model can be trained so that when the AI model is deployed, the system 200 can identify and predict one or more dimensions of the anatomical structure 420.

At 330, the system 200 acquires, at the display device 150, a new ultrasound image 402 during ultrasound scanning.

The display interface 410 shows a frozen image 402 comprising the anatomical structure 420 of interest (basilic vein) in transverse view. The frozen image 402 is the new ultrasound image that can be acquired when a user pauses the acquisition of ultrasound image frames by pressing a ‘freeze’ button 414. A selection of icons at the left of interface screen 410 includes an AI icon 412, which, once selected, activates identification and prediction of the one or more dimensions of the anatomical structure 420.

At 340, the system 200, using the AI model, processes the new ultrasound image 402 to identify and predict the one or more dimensions of the anatomical structure 420.

As described herein, such identification and prediction my be achieved by a variety of methods, including, but not limited to, segmentation of boundaries/edge detection, contouring and classification. This invention is not intended to be limited to any one mode of AI-model-generated anatomical structure identification. The product of the AI model is an output prediction as will be described, and/or a segmented anatomical structure for display on the screen 152.

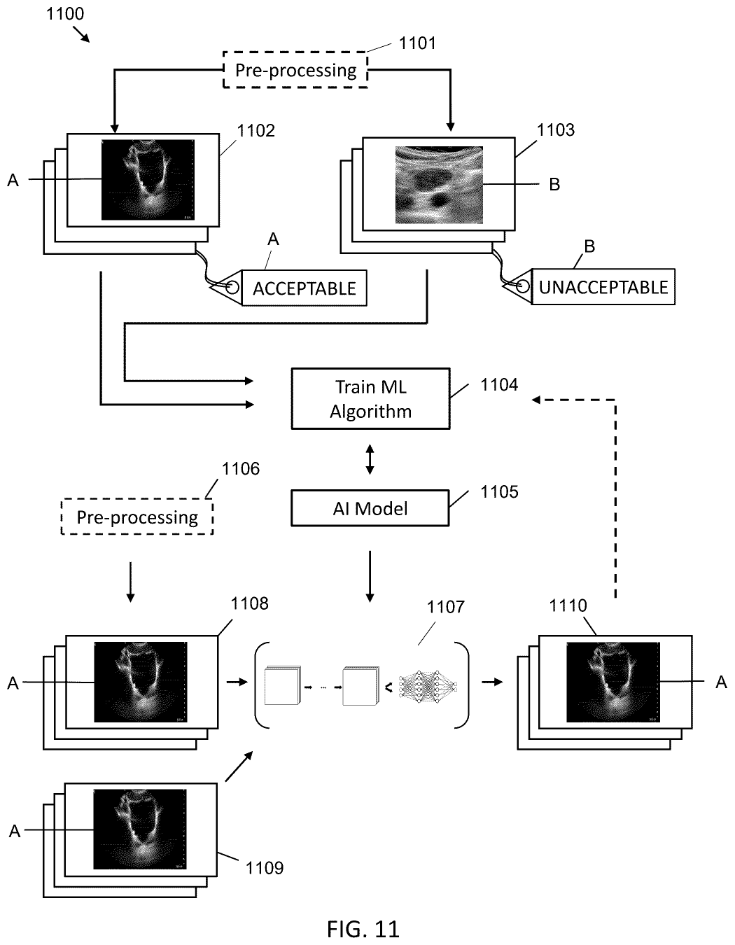

In various embodiments, a variety of means to segment the ultrasound image 402 may be used. For example, segmentation may be performed by dividing it into multiple parts or regions that belong to the same class. This task of clustering is based on specific criteria, for example, color or texture and is referred to as pixel-level classification. This involves partitioning images into multiple segments or objects using techniques including, but not limited to 1) thresholding, wherein a threshold value is set, and all pixels with intensity values above or below the threshold are assigned to separate regions; 2) region growing, wherein the ultrasound image 402 is divided into several regions based on similarity criteria. This segmentation technique starts from a seed point and grows the region by adding neighboring pixels with similar characteristics; 3) edge-based segmentation wherein segmentation techniques are based on detecting edges in the ultrasound image and these edges represent boundaries between different regions that are detected using edge detection algorithms; 4) clustering, wherein groups of pixels are clustered based on similarity criteria. These criteria can be color, intensity, texture, or any other feature; 5) active contours, also known as snakes, wherein curves that deform are used to find the boundary of an object in an image. These curves are controlled by an energy function that minimizes the distance between the curve and the object boundary; 6) deep learning-based segmentation, such as by employing Convolutional Neural Networks (CNNs), which employ a hierarchical approach to image processing, where multiple layers of filters are applied to the input image to extract high-level features, the training of which is described herein in FIG. 11.

FIG. 4B shows the ultrasound image 402 following deployment of the AI model for identifying and predicting dimensions of the anatomical structure 420. As can be seen, a general shape of the anatomical structure 420 has been identified with a visual indicator 430. FIG. 4C shows the ultrasound image 402 with another visual indicator 432 corresponding to the dimension of the anatomical structure 420, which, in this case is the transverse diameter of the basilic vein. Although FIGS. 4B and 4C show the visual indicators 430 and 432 in separate images, it will be understood that the system 200 can generate both visual indicators 430, 432 together, or, in some embodiments, no visual indicators 430, 432 can be provided. The visual indicators 430, 432 are merely shown herein to assist with the disclosure.

The one or more dimensions to be predicted can vary depending on various factors including, but not limited to, the clinical application, the anatomical structure itself, characteristics of the patient, etc. The dimensions can include, but not limited to, a diameter of the anatomical structure, a length of the anatomical structure, a width of the anatomical structure, circumference of the anatomical structure, an area of the anatomical structure, and/or a height of the anatomical structure. It is to be noted that when an anatomical structure is a vein, as opposed to an artery, the AI model of the invention is specifically trained to recognize, for the purpose of both segmentation (herein referred to intermittently as application of visual indicator) and of selection and placement on that segmented feature of dimension-determining caliper points, that veins are highly compressible and are rarely round in conformation, as shown on an ultrasound image. Shape of a vein is known to vary with external pressure, respiration, pressure from adjacent arteries, and even pressure from an ultrasound scanner itself over the skin. In other words, many veins are often close to the skin surface, so just the weight of the ultrasound scanner in operation can compress and distort them to a degree. This compression and shape distortion is shown in the jugular vein displayed in FIGS. 6A-6E. By way of comparison, this compression is less marked in arteries, such as the abdominal aorta shown in FIGS. 8A-8D, which presents, due to force of blood pressure therein, as a nearly rounded structure.

Similarly, wherein an anatomical feature is a trachea, as shown in FIGS. 7A-7E, segmentation and measurement acquisition, in accordance with one aspect of the present invention may be feature-specialized. While the trachea in an ultrasound image should present as a circle, it does not due air in the trachea (which is always present in living subjects) and, as such, the back wall of the trachea cannot be viewed in an ultrasound image instead presenting as a dirty shadow from the top down. For this feature specific reason, the AI model of the present invention may not necessarily segment the whole area in favour of a partial segmentation. Likewise, due to the superficiality of the trachea, determination of a point to calculate the cross-sectional diameter may be shallow, for example to 2.5-3.5 cm marker on the ultrasound image.

These and other anatomically specific characteristics are employed in the training and deployment of the AI model of the present invention including: i) in the selection of one or more dimension-determining caliper points/measurement points; and ii) using the one or more dimensions, so selected and measured, in the selection of a particular size of device, of the plurality of devices. For example, wherein the dimension is a diameter of a vein, the trained AI model and associated workflow of the present invention may select the smallest diameter available in the segmented area so as to avoid issues in selecting a catheter that is too large, and which may present other medical complications. The AI model of the invention may be trained to balance competing interests and risks. While it is beneficial to select a catheter that is small enough to fit in the vessel, it must be large enough to deliver the required medication. Too small may not be able to deliver a desired volume of medication and may pose a greater risk for thrombosis (particularly in larger veins like the jugular vein and the femoral vein). Too large may occasion trauma upon the vessel. Depending on the anatomical structure, a default device selection may be to select a device which is too small as opposed to too large. For some anatomical structures, the default may be the opposite. As such, additional and varied health considerations may be used to train the AI model of the present invention and may be used in the deployment of the AI model, including, but not limited to one or more of: i) characteristics of the anatomical structure; ii) characteristics of a health condition; iii) a clinical application; iv) device type and location sought to be placed; v) best practices for device placement; vi) historical records; and vii) patient age and other health considerations.

At 350, the system 200 automatically selects a device from the plurality of devices for placement therein based on the one or more dimensions.

The system 200 can apply the AI model to select the size of the device from a plurality of devices based on at least one of characteristics of the anatomical structure 420, the characteristics of a patient, a clinical application, best practices for device placement, and/or historical records.

Continuing with the example disclosed in FIGS. 4A to 4C, reference will now be made to FIG. 4D, which is a graphical representation 400D of automatically selecting a device for the anatomical structure 420. The anatomical structure 420 is the basilic vein, which is a typical location for a peripheral catheter. Generally, for catheters, the internal diameter of the anatomical structure 420 is important. Based on the internal diameter of the anatomical structure 420, the AI model can proceed to automatically select a size of the catheter with an external diameter that best fits the internal diameter of the anatomical structure 420. In FIG. 4D, for case of exposition, a plurality of catheter sizes 440a to 440e are shown. The AI model can select the catheter size that best aligns with the internal diameter determined at 340. In this case, the catheter size 440c was selected. It will be understood that FIG. 4D is illustrated for case of exposition and that the AI model can select the device without generating such an illustration. FIG. 4E shows the image 400E with the device 440c (peripheral catheter) overlaid onto the anatomical structure 420 for illustrative purposes. As can be seen, the device 440c is appropriately sized for the identified anatomical structure 420. The device overlay 440c shown in 400E is optional but can assist with the usage of the system 100/200. In some embodiments, the system 100/200 can generate an audio signal to indicate the selection of the device 440c (in additional to the visual overlay or alternative to that).

When the AI model identifies two different sizes of a device, the AI model can, by default, proceed to select a smaller size from the two different sizes. In some embodiments, the system 200 can set the AI model to adapt the selection based on various factors, including, but not limited to, characteristics of the anatomical structure, characteristics of a patient, a clinical application, best practices for device placement, and/or historical records.

In some cases, the AI model may identify a standardized size for the device based on the dimension of the anatomical structure 420. For example, catheters can be organized under a French catheter scale with each French catheter size associated with specific diameters. When the AI model is set up for identifying devices according to standardized sizes, the AI model can identify the standardized size and then select the size of the device that aligns with the standardized size.

Several other example applications of the methods and systems disclosed herein will be described with reference to FIGS. 5A to 8C.

FIG. 5A is an image 500A of the display interface 410 with another ultrasound image feed showing an anatomical structure 520. In this example, the ultrasound image 502 acquired includes a B-mode image of a region of interest. The region of interest includes an adductor canal 520 in longitudinal view. The adductor canal 520 is usually where a catheter can be placed to provide sensory blockade (e.g., nerve blockage for pain management) for as long as the catheter stays in place. FIG. 5B shows the image 500B following deployment of the AI model for identifying and predicting dimensions of the anatomical structure 520. The AI model generates the visual indicator 530 for identifying the general shape of the anatomical structure 520. The image 500C in FIG. 5C shows a further visual indicator 532 to identify a length of the illustrated portion of the anatomical structure 520 (adductor canal).

FIG. 6A is an image 600A of the display interface 410 with another ultrasound image feed showing an anatomical structure 620. The anatomical structure 620 in this example is a jugular vein. As shown in FIG. 6A, the ultrasound image 602 includes a B-mode image of a region of interest that includes the anatomical structure 620 (jugular vein in transverse view). Jugular veins are the location for central line placements (e.g., central venous catheter). FIG. 6B shows an image 600B following deployment of the AI model for identifying and segmenting the anatomical structure 620. The AI model generates the visual indicator 630 for identifying the general shape of the anatomical structure 620. The image 600C in FIG. 6C shows a further visual indicator 632 to represent a predicted diameter of the anatomical structure 620.

In FIG. 6D, a graphical representation 600D of automatically selecting a device for the anatomical structure 620 is shown. Based on the internal diameter of the anatomical structure 620, the AI model can proceed to automatically select a size of the catheter with an external diameter that best fits the internal diameter of the anatomical structure 620, which as shown in FIG. 6D, is catheter size 640c. In this example, a central line catheter is being selected for the jugular vein 620. For case of exposition, a plurality of catheter sizes 640a to 640d are shown. The AI model can select the catheter size that best aligns with the determined internal diameter. FIG. 6E shows the image 600E with the central line catheter 640c overlaid on the illustrated jugular vein 620, appropriately sized in accordance with at least one embodiment of the present invention.

FIG. 7A is an image 700A of a display interface 410 with an ultrasound image feed showing an anatomical structure 720. The ultrasound image 702 acquired is a B-mode image of a region of interest comprising a trachea 720. Endotracheal tubes are often inserted into tracheas to assist with airway support and/or offer access to the airway. FIG. 7B shows an image 700B following deployment of the AI model for identifying and segmenting the anatomical structure 720. The AI model generates the visual indicator 730 for identifying the general shape of the anatomical structure 720. The image 700C in FIG. 7C shows a further visual indicator 732 to represent a predicted diameter of the anatomical structure 720.

To select the device (e.g., endotracheal tube) for the anatomical structure 720, the AI model can consider dimensions of the internal diameter of the anatomical structure 720 (trachea). The AI model can then proceed to select the size of the device (endotracheal tube) based upon a measurement gauge of an external diameter of the endotracheal tube. FIG. 7D, for example, shows an example graphical representation of automatically selecting the device from a plurality 740 of devices for the anatomical structure 730. In this example, the AI model selects device size 740c for the anatomical structure 730 based on the dimension(s) determined. FIG. 7E shows an image 700E of the display interface 410 in which the ultrasound image 702 shows an overlay of the endotracheal tube sized as shown at 740c within the anatomical structure 730.

In some embodiments, the system 200 can apply the AI model to select the size of the endotracheal tube from multiple sizes, such as two different sized endotracheal tubes, based on one or more of a purpose of endotracheal tube placement, characteristics of the trachea, characteristics of a patient, a clinical application, best practices for endotracheal tube placement, and/or historical records.

FIG. 8A is an image 800A of the display interface 410 with another ultrasound image feed showing an anatomical structure 820. In this example, the ultrasound image 802 is a B-mode image of a region of interest comprising an abdominal aorta 820 in a transverse view. FIG. 8B shows the image 800B following deployment of the AI model for identifying and predicting dimensions of the anatomical structure 820. The AI model generates the visual indicator 830 for identifying the general shape of the anatomical structure 820. As described, the visual indicator 830 can be generated following various methods of segmentation of the ultrasound image 802. The image 800C in FIG. 8C shows a further visual indicator 832 to identify a predicted diameter of the illustrated portion of the anatomical structure 820 (abdominal aorta). In FIG. 8D, a graphical representation 800D of automatically selecting a device (here, a stent) for the anatomical structure 820 is shown. Based on the internal diameter of the anatomical structure 820, the AI model can proceed to automatically select a size of the stent with a diameter that best fits the diameter of the abdominal aorta 820, which as shown in FIG. 8D, is catheter size 840c. For case of exposition, a plurality of stent sizes 840a to 840e is shown. The AI model can select the stent size that best aligns with the determined diameter. FIG. 8D shows the image 800D with the stent 840c overlaid on the illustrated abdominal aorta 820, appropriately sized in accordance with at least one embodiment of the present invention.

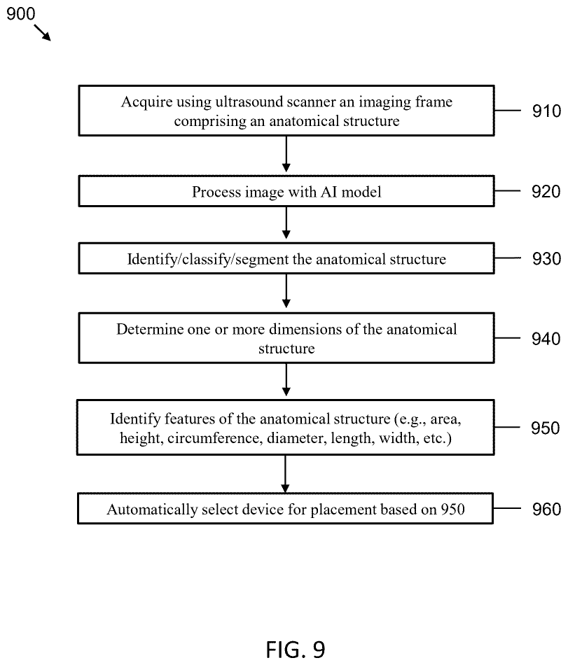

Referring now to FIG. 9, which shows a flowchart diagram of a method, generally indicated at 900, of selecting from a plurality of devices for placement within an anatomical structure on an ultrasound image feed acquired from the ultrasound scanner 102.

At 910, the system 200 acquires, using the ultrasound scanner 102, an imaging frame comprising an anatomical structure 420. Similar to 310 and 330 of FIG. 3 as described above.

At 920, the system 200 processes the image 402 with an AI model as described herein. For example, with respect to 320 and 340 of FIG. 3.

At 930, the system 200 identifies the anatomical structure 420. To identify the anatomical structure 420, the system 200 can classify and/or segment the image 402 in accordance with the methods and systems described herein.

At 940, the system 200 determines one or more dimensions of the anatomical structure 420. Similar to 340 of FIG. 3 as described above.

At 950, the system 200 identifies various features of the anatomical structure 420 (e.g., area, height, circumference, diameter, length, width, etc.). The system 200 can set the AI model to adapt the features to be identified based on various factors, including, but not limited to, characteristics of the anatomical structure, characteristics of a patient, a clinical application, best practices for device placement, and/or historical records.

At 960, the system 200 automatically selects device for placement based on 950. Similar to disclosure in respect of 350 and in respect of examples shown in FIGS. 5A to 8C.

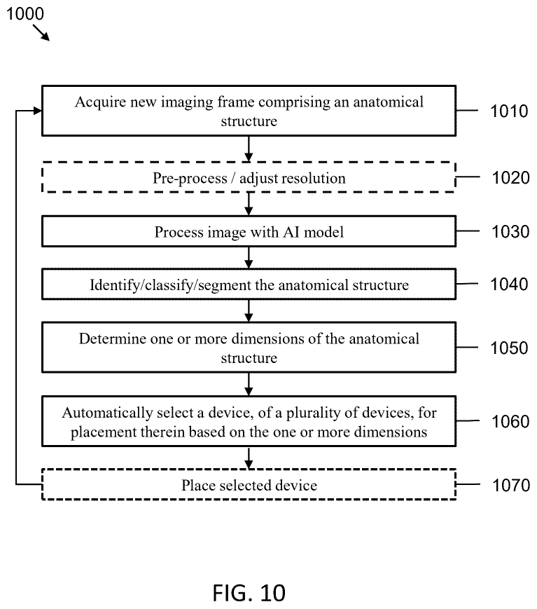

Referring now to FIG. 10, which shows a flowchart diagram of a method, generally indicated at 1000, of a usage of the methods and systems described herein for selecting from a plurality of devices for placement within an anatomical structure on an ultrasound image feed acquired from the ultrasound scanner 102.

At 1010, the system 200 acquires, using the ultrasound scanner 102, a new imaging frame 402 comprising the anatomical structure 420. Similar to 310 and 330 of FIG. 3 as described above.

At 1020, the system can pre-process resolution of the image 402.

This can include adjusting the resolution. For example, the resolution may be increased or decreased. Besides the resolution, other parameters of the ultrasound image 402 may also be adjusted such as input scaling, screen size, pixel size, aspect ratio, and the removal of dead space, as described above (including, for example, data augmentation and other preprocessing steps).

For example, it may be possible to pre-process the ultrasound imaging frame through a high contrast filter to reduce the granularity of greyscale on the ultrasound image 402. Additionally, or alternatively, it may be possible to reduce scale of the ultrasound image frame prior to providing the ultrasound image frame for processing through the AI model. Reducing the scale of ultrasound image frame as a preprocessing step may reduce the amount of image data to be processed, and thus may reduce the corresponding computing resources required. Various additional or alternative pre-processing acts may be performed. For example, these acts may include data normalization to ensure that the various ultrasound imaging frame has the dimensions and parameters which are optimal for processing through the AI model.

In some embodiments, these pre-processing acts may be to better align the ultrasound images 402 with the training ultrasound image frames, and thereby facilitate improved accuracy in feature segmentation. For example, pre-processing an input image 402 may help standardize the input image 402 so that it matches the format (e.g., having generally the same dimensions and parameters) of the training ultrasound images that the AI model is trained on.

At 1030, the system 200 processes the image 402 with an AI model. At 1040, the system 200 identifies the anatomical structure. To identify the anatomical structure, the system 200 can classify and/or segment the image as described herein.

At 1050, the system 200 determines one or more dimensions of the anatomical structure. Similar to the disclosure in respect of 320 of FIG. 3.

At 1060, the system automatically selects a device for placement based on 1050. Similar to the disclosure in respect of 350 and in respect of examples shown in FIGS. 5A to 8C.

At 1070, a user of the system 200 can then proceed to place the selected device into the anatomical structure 420. Method 1000 can repeat as necessary to improve placement and/or for purpose of placing other devices.

Referring to FIG. 11, shown therein generally at 1100 is a schematic diagram of a training and deployment of an AI model 1105. According to an embodiment of the present invention, there is shown a method of training a neural network 1107 so that when the AI model 1105 is deployed, a computing device identifies and segments boundaries of features, in whole or part. Specifically, during use and deployment, neural network 1107 identifies, in a new ultrasound image 402, an anatomical structure 420 and associated dimensions, in an ultrasound image frame.

For training, a number of ultrasound frames of a ROI (in whole view, from varying perspectives and parts thereof) may be acquired using the ultrasound scanner 102 (hereinafter “scanner”, “probe”, or “transducer” for brevity). The ultrasound frames may be acquired by scanning a series of planes (with a frame each containing a sequence of transmitted and received ultrasound signals), through an angle and capturing a different ultrasound frame at each of a number of different angles. During the scanning, the scanner 102 may be held steady by an operator of the scanner 102 while a motor in the head of the scanner tilts the ultrasonic transducer to acquire ultrasound frames at different angles. Additionally, or alternatively, other methods of acquiring a series of ultrasound frames may be employed, such as using a motor to translate (e.g., slide) the ultrasonic transducer or rotate it, or manually tilting, translating or rotating the ultrasound scanner.

The AI model 1105 is preferably trained with a robust selection of images of varying views. For example, these different views may include transverse plane views of a ROI, including views from different angles that combine any of a sagittal plane view, a coronal plane view, or a transverse plane view. In these embodiments, the scanner 102 may be placed in an arbitrary orientation with respect to the ROI, provided that the scanner 102 captures at least a portion of the ROI.

In some embodiments, ultrasound scans of a ROI, for training, may be acquired from medical examinations. During the scans, images may be obtained; however, for training of the AI model 1105 of the invention, non-clinically useful or acceptable images may also be used.

Referring still to FIG. 11, training ultrasound frames (1102 and 1103) may include ultrasound frames with features that are tagged as acceptable (A) and representative of images which are segmented and most advantageously boundaries of various anatomical structures or alternatively are tagged respectively as unacceptable (B) and unrepresentative of such division and segmentation. By way of example, in ultrasound frame 1102, which is marked as acceptable, there is provided an anatomical structure image which is marked as correctly and at least adequality segmented and identified. Conversely, ultrasound frame 1103, of the same ROI as ultrasound frame 1102, is marked as unacceptable, due to the fact that the features are unclear, and/or unclear and/or are at least non-adequality segmented.

Both the training ultrasound frames labeled as Acceptable and Unacceptable, for each particular ROI (whole or part), may themselves be used for training and/or reinforcing AI model 1105. As such, ultrasound frame 1103 may be employed for training as an unacceptable image.

In some embodiments, an optional pre-processing act 1101 may be performed on the underlying ultrasound image frames 1102 and 1103 to facilitate improved performance and/or accuracy when training the machine learning (ML) algorithm. For example, it may be possible to pre-process the ultrasound images 1102 and 1103 through a high contrast filter to reduce the granularity of greyscale on the ultrasound images 1102 and 1103.

Additionally, or alternatively, it may be possible to reduce scale of the ultrasound images 1102 and 1103 prior to providing the ultrasound images 1102 and 1103 to the training algorithm step 1104. Reducing the scale of ultrasound images 1102 and 1103 as a preprocessing step may reduce the amount of image data to be processed during the training act 1104, and thus may reduce the corresponding computing resources required for the training act 1104 and/or improve the speed of the training act 1104.

Various additional or alternative pre-processing acts may be performed in act 1101. For example, these acts may include data normalization to ensure that the various ultrasound frames 1102 and 1103 used for training have generally the same dimensions and parameters.

Referring still to FIG. 11, the various training frames 1102 and 1103 may, at act 1104, be used to train a ML algorithm. For example, the various training ultrasound frames 1102 and 1103, may be inputted into deep neural network 1107 that can learn how to predict boundaries of features in new ultrasound images as compared to all trained and stored images.

The result of the training may be the AI model 1105, which represents the mathematical values, weights and/or parameters learned by the deep neural network to predict segmented boundaries of features, within a ROI, in whole or part. The training act 1104 may involve various additional acts (not shown) to generate a suitable AI model 1105. For example, these various deep learning techniques such as regression, classification, feature extraction, and the like. Any generated AI models may be iteratively tested to ensure they are not overfitted and sufficiently generalized for creating the comparison and list of probabilities in accordance with method of the invention.

In some embodiments, using a cross-validation method on the training process would optimize neural network hyper-parameters to try to ensure that the neural network can sufficiently learn the distribution of all possible image types without overfitting to the training data. In some embodiments, after finalizing the neural network architecture, the neural network may be trained on all of the data available in the training image files.

In various embodiments, batch training may be used, and each batch may consist of multiple images, thirty-two for example, wherein each example image may be gray-scale, preferably 128*128 pixels although 256*256 pixels and other scaled may be used, without any preprocessing applied to it.

In some embodiments, the deep neural network parameters may be optimized using the Adam optimizer with hyper-parameters as suggested by Kingma, D. P., Ba, J. L.: Adam: a Method for Stochastic Optimization, International Conference on Learning Representations 2015 pp. 1-15 (2015), the entire contents of which are incorporated herewith. The weight of the convolutional layers may be initialized randomly from a zero-mean Gaussian distribution. In some embodiments, the Keras™ deep learning library with TensorFlow™ backend may be used to train and test the models.

In some embodiments, during training, many steps may be taken to stabilize learning and prevent the model from over-fitting. Using the regularization method, e.g., adding a penalty term to the loss function, has made it possible to prevent the coefficients or weights from getting too large. Another method to tackle the over-fitting problem is dropout. Dropout layers limit the co-adaptation of the feature extracting blocks by removing some random units from the neurons in the previous layer of the neural network based on the probability parameter of the dropout layer. Moreover, this approach forces the neurons to follow overall behaviour. This implies that removing the units would result in a change in the neural network architecture in each training step. In other words, a dropout layer performs similar to adding random noise to hidden layers of the model. A dropout layer with the dropout probability of 0.5 may be used after the pooling layers.

Data augmentation is another approach to prevent over-fitting and add more transitional invariance to the model. Therefore, in some embodiments, the training images may be augmented on-the-fly while training. In every mini-batch, each sample may be translated horizontally and vertically, rotated and/or zoomed, for example. The present invention is not intended to be limited to any one particular form of data augmentation, in training the AI model. As such, any mode of data augmentation which enhances the size and quality of the data set and applies random transformations which do not change the appropriateness of the label assignments may be employed, including but not limited to image flipping, rotation, translations, zooming, skewing, and elastic deformations.

Referring still to FIG. 11, after training has been completed, the sets of parameters stored in the storage memory may represent a trained neural network of a plurality of images of ROIs which identifies and segments boundaries of features with each ROI, in whole or part.

In order to assess the performance of AI model 1105, the stored model parameter values can be retrieved any time to perform image assessment through applying an image to the neural networks (shown as 1107) represented thereby. In some embodiments, the deep neural network may include various layers such as convolutional layers, pooling layers, and fully connected layers. In some embodiments, the final layers may include a softmax layer as an output layer having outputs which eventually would demonstrate respective determinations that an input set of pixels fall within a particular area above or below a feature boundary, in the training images. Accordingly, in some embodiments, the neural network may take at least one image as an input and output a binary mask indicating which pixels belong to the area above a feature boundary (or part thereof), e.g., the AI model classifies which area each pixel belongs to.

To increase the robustness of the AI model 1105, in some embodiments, a broad set of training data may be used at act 1104. For example, it is desired that ultrasound images of a plurality of different ROIs, across a plurality of anatomical regions in a body, in whole and a variety of parts thereof, from views including but not limited to coronal and/or transverse plane views, including views from different angles that combine any of a sagittal plane view, a coronal plane view, or a transverse plane view.

More specifically, training images 1102 and 1103 may be labeled with one or more features associated with/are hallmarks of a particular ROI, including key anatomical features therein. This may include identifying a variety of features visualized in the captured training image. In at least some embodiments, this data may be received from trainer/user input. For example, a trainer/user may label the features relevant for the application visualized in each training image.

The image labeling can be performed, for example, by a trainer/user observing the training ultrasound images, via a display screen of a computing device, and manually annotating the image via a user interface. In some aspects, the training ultrasound images used for the method herein will only be images in which the image quality is of a sufficient quality threshold to allow for proper and accurate feature identification. For example, this can include training ultrasound images having a quality ranging from a minimum quality in which target features are just barely visible for labelling (e.g., annotating), to excellent quality images in which the target features are easily identifiable. In various embodiments, the training medical images can have different degrees of image brightness, speckle measurement and SNR. Accordingly, training ultrasound images 1102 and 1103 can include a graduation of training images ranging from images with just sufficient image quality to high image quality. In this manner, the machine learning model may be trained to identify features on training medical images that have varying levels of sufficient image quality for later interpretation and probability assessment.

Overall, the scope of the invention and accorded claims are not intended to be limited to any one particular process of training AI model 1105. Such examples are provided herein by way of example only. AI model 1105 may be trained by both supervised and unsupervised learning approaches although due to scalability, unsupervised learning approaches, which are well known in the art, are preferred. Other approaches may be employed to strengthen AI model 1105.

The image labelling can be performed, for example, by a trainer/user observing the training ultrasound images, via a display screen of a computing device, and manually annotating the image via a user interface. In some aspects, the training ultrasound images used for the method herein will only be images in which the image quality is of a sufficient quality threshold to allow for proper and accurate feature identification. For example, this can include training ultrasound images having a quality ranging from a minimum quality in which target features are just barely visible for labelling (e.g., annotating), to excellent quality images in which the target features are casily identifiable. In various embodiments, the training medical images can have different degrees of image brightness, speckle measurement and SNR. Accordingly, training ultrasound images can include a graduation of training medical images ranging from images with just sufficient image quality to high image quality. In this manner, the machine learning model may be trained to identify features on training medical images that have varying levels of sufficient image quality for later interpretation and probability assessment.

Turning back to FIG. 11, once a satisfactory AI model 1105 is generated, the AI model 1105 may be deployed for execution on a neural network 1107 to identify and segment boundaries of features, in whole or part, within a ROI. Notably, the neural network 1107 is shown in FIG. 11 for illustration as a convolution neural network—with various nodes in the input layer, hidden layers, and output layers. However, in various embodiments, different arrangements of the neural network 1107 may be possible.

The training image file may include an image identifier field for storing a unique identifier for identifying an image included in the file, a segmentation mask field for storing an identifier for specifying the to-be-trimmed area, and an image data field for storing information representing the image.

Referring again to FIG. 11, once a satisfactory AI model 1105 is generated, the AI model 1105 may be deployed for execution on a neural network 1107 to segment the anatomical structure, as described fully herein, new ultrasound images 1108. Notably, the neural network 1107 is shown in FIG. 11 for illustration as a convolution neural network—with various nodes in the input layer, hidden layers, and output layers. However, in various embodiments, different arrangements of the neural network 1107 may be possible.

In various embodiments, the new ultrasound images 1108 may be live images acquired by an ultrasound imaging system 100, 200 (e.g., the system discussed with respect to FIGS. 1 and 1). For example, the AI model 1105 may be deployed for execution on the scanner 102 and/or the display device 150 discussed herein. Additionally, or alternatively, the AI model 1105 may be executed on stored (as opposed to new) ultrasound images 1109 that were previously acquired (e.g., as may be stored on a Picturing Archiving and Communication System (PACS)).

Whether the images are stored ultrasound images 1109 or new ultrasound images 1108, the AI model 1105 enables the neural network 1107 to properly segment a feature within a ROI imaged in the new/stored ultrasound imaging data and created an identified and segmented image frame 1110.

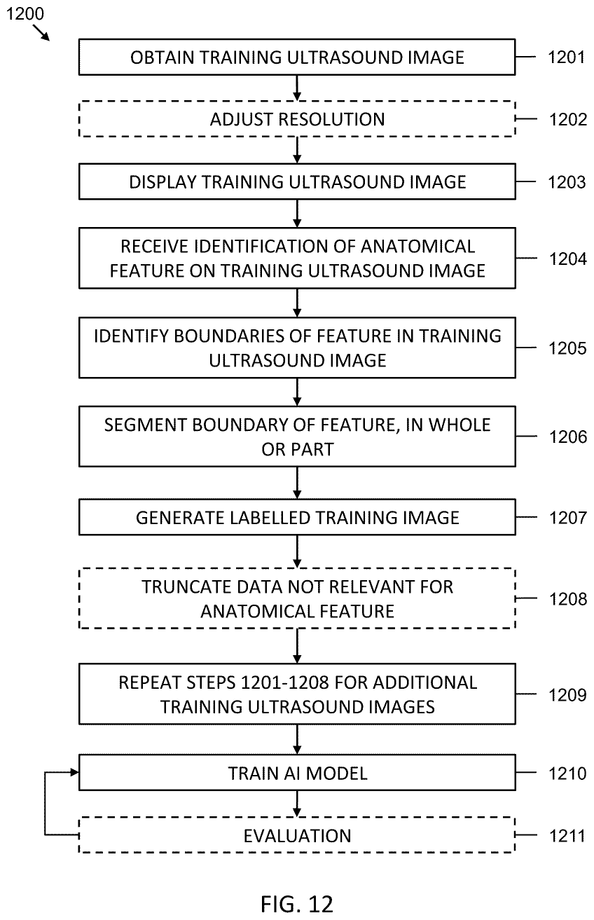

FIG. 12 is flowchart diagram of the steps, generally indicated as 1200, for training the AI model 1105 of FIG. 11, according to an embodiment of the present invention. In some embodiments, method 1200 may be implemented as executable instructions in any appropriate combination of the imaging system 100 (FIG. 1), for example, an external computing device connected to the imaging system 100, in communication with the imaging system 100, and so on. As one example, method 1200 may be implemented in non-transitory memory of a computing device, such as the controller (e.g., processor) of the imaging system 100.

Referring still to FIG. 12, in step 1201, a training ultrasound image may be obtained. For example, a training ultrasound image may be acquired by the scanner 102 (as shown in FIG. 1) transmitting and receiving ultrasound energy. The training ultrasound image may generally be a post-scan converted ultrasound image. While the method of FIG. 12 is described in relation to a single training ultrasound image, the method may also apply to the use of multiple training ultrasound images. While the method of FIG. 12 is described in relation to a post-scan ultrasound image, it is to be understood that pre-scan images, may be used, as described in U.S. patent application Ser. No. 17/187,851 filed Feb. 28, 2021, the entire contents of which are incorporated herein by reference.

Optionally, in step 1202 (as shown in dotted outline), the resolution of the training ultrasound image may be adjusted. For example, the resolution may be increased or decreased. The purpose of this may be to provide the labeler (e.g., a medical professional with relevant clinical expertise) with training ultrasound images that have a more standardized appearance. This may help to maintain a higher consistency with which the labeler identifies anatomical features in the training ultrasound images. Besides the resolution, other parameters of the training ultrasound image may also be adjusted such as input scaling, screen size, pixel size, aspect ratio, and the removal of dead space, as described above (including, for example, data augmentation and other preprocessing steps).

In step 1203, the training ultrasound image may be displayed on a display device, such as the display device 150 discussed in more detail in relation to FIG. 1. The labeler can then identify a particular anatomy in the training ultrasound image by, for example, tagging it with a name from a pull-down menu or by using other labeling techniques and modalities. The labeler then can mark the training ultrasound image around the particular anatomy that the labeler has identified in the training ultrasound image. In step 1204, the system that is used for the training may receive the identification of the anatomical feature(s) on the training ultrasound image. In step 1205, the system may generate, for example, from a labeler's marking inputs, identified boundaries of a feature or features in the training ultrasound frame. In step 1206, a boundary feature is segmented, and one or more dimensions of the anatomical structure are identified in order to, at step 1207, generate a labeled training image.

In various embodiments, steps may readily be interchanged with each other. For example, the generation of labeled confirmation at step 1207 may automatically proceed, without trainer intervention, using prior data which directs to the placement of feature boundaries.

Once the training ultrasound image has been segmented and labeled, the system may then remove, in step 1208, optionally, (as shown in dotted outline), regions of the labeled ultrasound data frame that are both outside the area of the identified boundary features and outside areas relevant for the AI model to recognize the particular anatomy within the ROI. For example, the labeled ultrasound data frame may be truncated at one or more sides. Truncation of some of the ultrasound data may allow the training of the AI model to proceed more quickly. There is provided a redirection at step 1209 to repeat steps 1201-1208 a plurality of times, for additional training images. At step 1210, AI model is trained. At step 1211, once training is completed, the AI model may be used to perform identifications and selections on an unseen dataset to validate its performance, such evaluation at step 1211 feeding data back to train the AI model at step 1210.

### D. Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Unless the context clearly requires otherwise, throughout the description and the claims:

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

For example, while processes or blocks are presented in a given order herein, alternative examples may perform routines having steps, or employ systems having blocks, in a different order, and some processes or blocks may be deleted, moved, added, subdivided, combined, and/or modified to provide alternative or sub combinations. Each of these processes or blocks may be implemented in a variety of different ways. Also, while processes or blocks are at times shown as being performed in series, these processes or blocks may instead be performed in parallel or may be performed at different times.

The invention may also be provided in the form of a program product. The program product may comprise any non-transitory medium which carries a set of computer-readable instructions which, when executed by a data processor (e.g., in a controller and/or ultrasound processor in an ultrasound machine), cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

To aid the Patent Office and any readers of any patent issued on this application in interpreting the claims appended hereto, applicant wishes to note that they do not intend any of the appended claims or claim elements to invoke 35 U.S.C. 112 (f) unless the words “means for” or “step for” are explicitly used in the particular claim.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples but should be given the broadest interpretation consistent with the description as a whole.

What is claimed is:

1. A method of selecting from a plurality of devices for placement within an anatomical structure on an ultrasound image feed that is acquired from an ultrasound scanner, the method comprising:
displaying, on a screen communicatively connected to the ultrasound scanner, the ultrasound image feed comprising the anatomical structure;
deploying an AI model to execute on a computing device communicatively connected to the ultrasound scanner, wherein the AI model is trained so that when the AI model is deployed, the computing device identifies and predicts one or more dimensions of the anatomical structure;
acquiring, at the computing device, a new ultrasound image during ultrasound scanning;
processing, using the AI model, the new ultrasound image to identify and predict the one or more dimensions of the anatomical structure; and
automatically selecting a device from the plurality of devices for placement therein based on the one or more dimensions.

2. The method of claim 1 further comprises:
applying the AI model to segment boundaries of the anatomical structure in the new ultrasound image, and
generating a segmented anatomical structure for display on the screen.

3. The method of claim 1 wherein the one or more dimensions is selected from the group consisting of a diameter of the anatomical structure, a length of the anatomical structure, a width of the anatomical structure, circumference of the anatomical structure, an area of the anatomical structure, and a height of the anatomical structure.

4. The method of claim 1, wherein the screen is within a multi-purpose electronic device which is communicatively coupled with the ultrasound scanner and an additional step of indicating the device, which is automatically selected, is via at least one of a visual signal on the display or an audio signal.

5. The method of claim 1 further comprises:
applying the AI model to identify a diameter of the anatomical structure; and
applying the AI model to automatically select the device for placement based on the diameter.

6. The method of claim 5 wherein more than one device is selected by the AI model based on the diameter of the anatomical structure, and an additional step comprises the AI model selecting a preferred device, of the more than one device, based upon a clinical application.

7. The method of claim 1 further comprises:
applying the AI model to select the size of the device from a plurality of devices based on at least one of i) characteristics of the anatomical structure; ii) characteristics of a patient; iii) a clinical application; iv) best practices for device placement; and v) historical records.

8. The method of claim 7 wherein the AI model i) identifies two devices of two different sizes from the plurality of devices, and ii) selects a smaller size from the two different sizes.

9. The method of claim 1 which further comprises:
identifying a standardized size for the device based on the one or more dimensions of the anatomical structure; and
selecting the size of the device that corresponds to the standardized size.

10. The method of claim 1 wherein the device is selected from the group consisting of a catheter, endotracheal tube and an implant.

11. The method of claim 10 wherein the device is a catheter, the one of more dimensions is an internal diameter of the anatomical structure and a size of the catheter is automatically selected by the AI model, based upon a measurement gauge of an external diameter of the catheter, as compared to a best fit of the internal diameter of the anatomical structure.

12. The method of claim 10 wherein the device is an endotracheal tube, the one of more dimensions is an internal diameter of a trachea and a size of the endotracheal tube is automatically selected by the AI model, based upon a measurement gauge of an external diameter of the endotracheal tube.

13. The method of claim 12 which further comprises:
applying the AI model to select the size of the endotracheal tube from two different sized endotracheal tubes based on at least one of: i) purpose of endotracheal tube placement; ii) characteristics of the trachea; iii) characteristics of a patient; iv) a clinical application; v) best practices for endotracheal tube placement; and vi) historical records.

14. The method of claim 10 wherein the implant is selected from the group consisting of spinal implants, orthopedic implants, neurological implants, vascular implants, and cardiac implants.

15. The method of claim 1 wherein the AI model is trained with a plurality of training ultrasound images comprising labelled segmented boundaries of the anatomical structure, in plurality of views, which are, one of: i) generated by one of a manual or semi automatic means; or ii) tagged from an identifier menu by one of a manual, semi automatic means or fully automatic means.

16. The method of claim 1 comprising training the AI model with one or more of the following:
i) supervised learning; ii) unsupervised learning; iii) previously labelled ultrasound image datasets; and iv) cloud stored data.

17. A system for selecting a plurality of devices for placement within an anatomical structure on an ultrasound image frame, the system comprising:
an ultrasound scanner configured to acquire the ultrasound image frame of the anatomical structure;
a display device communicatively connected to the ultrasound scanner, the display device comprising a screen configured to display the ultrasound image frame; and
a computing device communicatively connected to the ultrasound scanner and configured to:
process the ultrasound image frame against an AI model trained to identify and predict one or more dimensions of the anatomical structure; and
automatically select a device from the plurality of devices for placement therein based on the one or more dimensions.

18. The system of claim 17 wherein the computing device is further configured to:
apply the AI model to identify a diameter of the anatomical structure; and
apply the AI model to automatically select the device for placement based on the diameter.

19. The system of claim 17 wherein the computing device is further configured to:
identify a standardized size for the device based on the one or more dimensions of the anatomical structure; and
select the size of the device that corresponds to the standardized size.

20. A computer-readable medium storing computer-readable instructions, which, when executed by a processor cause the processor to:
display, on a screen communicatively connected to the ultrasound scanner, the ultrasound image feed comprising the anatomical structure;
deploy an AI model to execute on a computing device communicatively connected to the ultrasound scanner, wherein the AI model is trained so that when the AI model is deployed, the computing device identifies and predicts one or more dimensions of the anatomical structure;
acquire, at the computing device, a new ultrasound image during ultrasound scanning;
process, using the AI model, the new ultrasound image to identify and predict the one or more dimensions of the anatomical structure; and
automatically select a device from the plurality of devices for placement therein based on the one or more dimensions.
